(19) United States
(12) Patent Application Publication
(10) Pub. No.: US 2024/0400823 A1
(43) Pub. Date: Dec. 5, 2024

(54) POLYMER COMPOSITES, METHODS OF FABRICATION AND USES THEREOF

(71) Applicant: National University of Singapore, Singapore (SG)

(72) Inventors: Jianyong OUYANG, Singapore (SG); Lei ZHANG, Singapore (SG); Hongliang REN, Singapore (SG); Kirthika SENTHIL KUMAR, Singapore (SG)

(73) Assignee: National University of Singapore, Singapore (SG)

(21) Appl. No.: 17/769,851

(22) PCT Filed: Oct. 16, 2020
(86) PCT No.: PCT/SG2020/050590

§ 371 Date: Apr. 18, 2022

(30) Foreign Application Priority Data
Oct. 18, 2019 (SG) 10201909714S

(51) Int. Cl. C08L 75/04; C08K 5/053

(52) U.S. Cl. CPC C08L 75/04; C08K 5/053; C08L 2201/54; C08L 2203/02; C08L 2203/20; C08L 2205/035

(57) ABSTRACT

The invention relates to a polymeric composite comprising poly(ethylenedioxythiophene):poly(styrenesulfonate) (PEDOT:PSS), waterborne polyurethane (WPU) and a sugar alcohol, wherein the sugar alcohol is selected from the group consisting of glycerol, erythritol, threitol, arabitol, xylitol, ribitol, mannitol, sorbitol, galactitol, fucitol, iditol, inositol, volemitol, isomalt, maltitol, lactitol, maltotriitol, maltotetraitol, polyglycitol, or a combination thereof, and wherein the sugar alcohol is 20 wt % to 50 wt % of the polymer composite; an electrical device comprising such composite. The present invention also relates to methods of fabricating and its uses thereof. In particular, the present invention relates to intrinsically conductive polymer composites suitable for use in efficient dry/wet epidermal biopotential monitoring. The polymer composites are advantageously self-adhesive and stretchable, and can be used as dry electrodes.

The present application is a national phase application under 35 U.S.C. § 371 of International Application No. PCT/SG2020/050590, filed Oct. 16, 2020, the entire contents of which are hereby incorporated by reference, and which claims the priority benefit of Singaporean Application No. 10201909714S, filed Oct. 18, 2019.

### FIELD OF INVENTION

The invention relates generally to a polymeric composition and an electrical device comprising such a polymeric composition. The present invention also relates to methods of fabricating and its uses thereof. In particular, the present invention relates to intrinsically conductive polymer composites suitable for use in efficient dry/wet epidermal biopotential monitoring. The polymer composites are advantageously self-adhesive and stretchable, and can be used as dry electrodes.

### BACKGROUND

Human biopotentials such as electrocardiography (ECG), electromyography (EMG), and electroencephalography (EEG) are significant for diagnosis and treatment of heart-, brain-, and muscle-related diseases. These biopotentials can be transduced by electrically interfacing with the skin via epidermal electrodes. An efficient wearable electrode is crucial for accurately recording these biopotential signals, especially in the case of continuous monitoring of inconspicuous heart diseases and rehabilitation in daily life. At present, Ag/AgCl gel electrodes are predominant in a clinical setting to obtain surface biopotentials, but prone to signal degradation in the long run of continuous monitoring due to the volatilization of the liquid in gel electrolyte and skin irritation. Further, although Ag/AgCl gel electrodes can give rise to high-quality signals, they are not suitable for use as wearable and long-term monitoring devices because of the evaporation of the liquid in the gel electrolyte.

Effort has been devoted to the development of skin-friendly dry electrodes for biopotential measurements. The dry electrodes currently in market can be classified mainly into dry contact electrodes and dry capacitive (noncontact) electrodes. The dry capacitive electrodes give rise to motion artefacts and are quite sensitive to body movement, and thus are not suitable for biopotential monitoring. The dry contact electrodes mainly include thin metal films, conductive polymers composites, and intrinsically conductive polymers. Although thin metal films can have high conductivity, they are not stretchable and adhesive. As a result, high noise can be observed on the biopotential signals, particularly during body movement.

Recent work on dry contact electrodes have been focused on soft conductive polymer composites and intrinsically conductive polymers due to their adaptation to rough and even deformed skin. The conductive polymer composites consist of elastomers and conductive nanofillers like metals, nanotubes, nanowires, and nanosheets. The conductive nanofillers are the minority in the elastomer matrix, leading to a small effective contact area between the conductive nanofillers and human skin. As a result, the electrode-skin interface impedance is higher than that with Ag/AgCl gel electrode by a couple of orders in magnitude, and significant effect can be observed on the biopotential signals. Mismatching between a dry electrode and human skin can occur during body movement, which can be improved if the dry electrodes are adhesive to human skin. Polymer composite patches with bio-inspired micropillar or sucker-like structures can be stretchable and adhesive. Nevertheless, their adhesion to the skin is easily affected by secreted sweat or dirt on the skin, and clustering or contamination of the structures. In addition, the suction induced adhesion of these structures can cause discomfort to patients. There is also concern about the toxicity of the nanofillers.

Accordingly, wearable dry biopotential electrodes for high quality recording are required for healthcare, particularly for long-term biomedical monitoring. They should have low impedance on skin so that biomedical signal with high signal-to-noise ratio can be obtained. In addition, they should be self-adhesive and stretchable so that they can adapt well on skin even during body movement.

Accordingly, it is generally desirable to overcome or ameliorate one or more of the above mentioned difficulties.

### SUMMARY

The present invention is predicated on the understanding that wearable dry electrodes are needed for long-term biopotential recordings but are limited by their imperfect compliance with the skin, especially during body movements and sweat secretions, resulting in high interfacial impedance and motion artifacts. To this end, the inventors have invented an intrinsically conductive polymer composite for use as a dry electrode with excellent self-adhesiveness, stretchability, and conductivity. The polymer composite shows much lower skin-contact impedance and noise in static and dynamic measurement than the current dry electrodes and standard gel electrodes, enabling high-quality electrocardiogram (ECG), electromyogram (EMG) and electroencephalogram (EEG) signals to be acquired in various conditions such as dry and wet skin and during body movement. The dry electrode can be used for long-term healthcare monitoring in complex daily conditions. Further investigations on the capabilities of this electrode in a clinical setting revealed that the dry electrode can detect the arrhythmia features of atrial fibrillation accurately, and can quantify muscle activity during deep tendon reflex testing and contraction against resistance. Similar tests done on glass also show that the polymer composites can properly adhere to a dry or wet surface.

The present invention provides a polymer composite, comprising:
a) poly(ethylenedioxythiophene):poly(styrenesulfonate) (PEDOT:PSS);
b) waterborne polyurethane (WPU); and
c) a sugar alcohol;
   wherein the sugar alcohol is selected from the group consisting of glycerol, erythritol, threitol, arabitol, xylitol, ribitol, mannitol, sorbitol, galactitol, fucitol, iditol, inositol, volemitol, isomalt, maltitol, lactitol, maltotriitol, maltotetraitol, polyglycitol, or a combination thereof; and
   wherein the sugar alcohol is about 20 wt % to about 50 wt % of the polymer composite.

In some embodiments, a ratio of PEDOT to PSS is about 2.5:1 w/w.

In some embodiments, PEDOT:PSS is about 4 wt % to about 25 wt % of the polymer composite.

In some embodiments, WPU is about 37 wt % to 58 wt % of the polymer composite.

In some embodiments, the sugar alcohol is about 38 wt % of the polymer composite.

In some embodiments, the sugar alcohol is D-sorbitol.

In some embodiments, the polymer composite further comprises ethylene glycol at about 0.2 wt % to 1.2 wt % of the polymer composite.

In some embodiments, the polymer composite comprises a homogenous blend of PEDOT:PSS, WPU and sugar alcohol, wherein PEDOT:PSS and WPU each form separate continuous phases in the polymer composite.

In some embodiments, when PEDOT:PSS loading is about 19 wt % of the polymer composite, the polymer composite has an elongation at break is about 35% to about 50%.

In some embodiments, a conductivity of the polymer composite is about 60 S/cm to about 600 S/cm.

In some embodiments, the polymer composite is repeatedly stretchable for at least 400 cycles.

In some embodiments, the polymer composite has a stretchability of more than about 40%.

In some embodiments, the polymer composite has an adhesion force to a skin of about 0.35 N/cm to about 0.7 N/cm.

In some embodiments, the polymer composite has an adhesion force to a glass surface of about 1 N/cm to about 2 N/cm.

The present invention also provides a polymer composite comprising:
a) poly(ethylenedioxythiophene):poly(styrenesulfonate) (PEDOT:PSS) at a ratio of about 2.5:1 w/w;
b) waterborne polyurethane (WPU); and
c) D-sorbitol;
   wherein (PEDOT:PSS) is about 4 wt % to about 25 wt % of polymer composite; and
   wherein the sugar alcohol is about 20 wt % to about 50 wt % of the polymer composite.

The present invention also provides an electrical device comprising a polymer composite as disclosed herein.

In some embodiments, the electrical device has an electrode-skin electrical impedances at 10 Hz of about 70 KΩ cm² to about 100 KΩ cm².

In some embodiments, the electrical device can generate an electrocardiogram (ECG) signal, wherein an ECG peak-to-peak voltage is about 1.6 mV to about 2 mV.

In some embodiments, the electrical device can generate an electromyogram (EMG) signal, wherein a peak-to-peak amplitude is linearly correlated to an applied force, and wherein a signal intensity is linearly correlated to the applied force.

In some embodiments, the electrical device can generate an electroencephalogram (EEG) signal, wherein the EEG signal perturbable by stimulating an optic nerve of a subject and/or an auditory stimuli.

The present invention also provides a method of preparing or fabricating a polymer composite, comprises:
a) mixing PEDOT:PSS with a sugar alcohol to form a first mixture;
b) mixing the first mixture with WPU to form a second mixture; and
c) curing the second mixture in order to form the polymer composite;
   wherein the sugar alcohol is selected from the group consisting of glycerol, erythritol, threitol, arabitol, xylitol, ribitol, mannitol, sorbitol, galactitol, fucitol, iditol, inositol, volemitol, isomalt, maltitol, lactitol, maltotriitol, maltotetraitol, polyglycitol, or a combination thereof; and
   wherein the sugar alcohol is about 20 wt % to about 50 wt % of the polymer composite.

### BRIEF DESCRIPTION OF DRAWINGS

Embodiments of the present invention are hereafter described, by way of non-limiting example only, with reference to the accompanying drawings in which:

FIG. 1 shows (A) Stress/strain curves; (B) conductivity of polymer composites with different PEDOT:PSS loadings; and (C) variations of the elongation at break and Young's modulus of the polymer composites with the PEDOT:PSS loading;

FIG. 2 shows a plot of adhesion force of a polymer composite on glass slide and dry skin;

FIG. 3 shows recorded ECG signals using commercial Ag/AgCl gel electrode (left) and prepared PEDOT electrode (right);

FIG. 4 shows recorded EMG signal of bicipital muscle during contraction and releasing;

FIG. 5 is a schematic illustration for the preparation of an exemplary polymer composite;

FIG. 6 shows some characterization and mechanical properties of an exemplary polymer composite;

FIG. 7 is an energy-dispersive X-ray (EDX) analysis of a polymer composite showing nitrogen (N) and sulfur (S) in the surface of polymer composite with PEDOT:PSS loading 19 wt %;

FIG. 8 shows mechanical properties of comparative polymer composite without sugar alcohol at various conductive polymer loading;

FIG. 9 shows the electrical properties of polymer composites;

FIG. 10 shows conformability and adhesiveness of polymer composites;

FIG. 11 shows an impedance spectra of an electrode on the skin a) with different thicknesses; b) at 10, 100 and 1000 Hz; c) placement of two PWS electrodes on the skin for the impedance measurements;

FIG. 12 a) is a schematic illustration of the ECG detection; b) is photos of electrodes attached firmly to the skin and then peeled off after 16 h; c) is comparison of ECG signals using an electrode and commercial Ag/AgCl gel electrode; d) is a spectrogram of the ECG pulse recorded using the PWS dry electrode; e) is long-term monitoring of ECG using PWS dry electrodes for 1 day and their RMS noise; f) is the RMS noise picked by Ag/AgCl gel electrode and PWS dry electrode during ECG recording in one-time, 1-day, and 1 week; g, h) is ECG testing on the skin under motion induced by an electrical vibrator. The distance of the vibrator from the electrode was 5, 3, or 1 cm;

FIG. 13 shows RMS noise produced by adhesive PWS electrodes, slight-adhesive PW electrode and non-adhesive PEDOT:PSS film electrode on the skin under motion induced by an electrical vibrator. The separation of the vibrator from the electrode is 5, 3, and 1 cm;

FIG. 14 shows a) Monitoring of the EMG signal on a forearm gripping a ball with different moduli of 0.21, 0.27, and 0.33 GPa, respectively. B) EMG signals while gripping the balls. C) Variations of the EMG signal amplitude and the gripping force with the modulus of the balls. D) Using EMG signals to control the motion of a robotic hand, including opening and closing. E) EMG signals produced by the flexion/extension of different fingers. F) EMG signal intensities produced by the five fingers;

FIG. 15 shows a) Fabrication of the 3D PWS electrodes. B) Photo of a 3D PWS electrode. C) Positioning two 3D PWS electrodes at the 01 and 02 sites of the rear head and a PWS film electrode behind the ear as the reference electrode. D) EEG signals collected during eye-blinking. E) EEG signals respond to auditory stimuli; and

FIG. 16 shows a) ECG signals showing the variability in the R-R intervals and absent P-waves, which are diagnostic of atrial fibrillation. B) EMG signals showing a brief and significant increase in muscle potentials detected using the PSW dry electrode by tapping on the biceps tendon. C) EMG signals showing incremental potentials during contraction of the biceps muscles, which decreased following relaxation.

### DETAILED DESCRIPTION

The present invention is based on the understanding that intrinsically conductive polymers can have high effective contact areas with human skin, biocompatibility, high electrical conductivity, and inherent mechanical flexibility. Poly(ethylenedioxythiophene):poly(styrenesulfonate) (PEDOT:PSS) can be used as dry electrodes. For example, PEDOT:PSS films printed on paper or polyimide foil can be used as the ECG dry electrodes. However, the signal has poor quality and the electrodes may delaminate from the skin, because the PEDOT:PSS films are not adhesive and stretchable. Further, while an earlier study reported that ultrathin films of ethyl cellulose/PEDOT:PSS bilayer can be adhesive to skin and be used as the EMG dry electrode, it was found that EMG signal is susceptible to strain during muscle movement because of the limited stretchability of ethyl cellulose/PEDOT:PSS bilayer. In addition, it is very difficult to handle ultrathin films. The inventors have postulated that to obtain high-quality biopotential signals, a dry electrode should have at least one of the following features: conductive, biocompatible, stretchable, conformable, and self-adhesive to the skin. Current intrinsically conductive polymers are neither stretchable nor adhesive to the skin.

The inventors have found that a polymer composite of an electrically conductive polymer, an elastomer and a sugar alcohol can be used as a dry electrode. When formed as a dry electrode, it is a fully-organic, self-adhesive, and stretchable dry electrode with high conductivity. It also possesses high conductivity and skin-compliant stretchability, with appreciated adhesion on dry and wet skin conditions, respectively. The dry electrode shows lower contact impedance on the skin and much lower noise level in static and dynamic detection than other dry electrodes in literature and standard Ag/AgCl gel electrodes. This dry electrode can always give rise to high-quality epidermal biopotential signals, including ECG, EMG, and EEG, in various conditions such as dry and wet skin and during body movement. Moreover, this dry electrode can precisely identify the arrhythmia of a patient with atrial fibrillation and muscle activity in a clinical setting. The polymer composite and/or dry electrode can be fabricated by solution processing of these biocompatible blends or ingredients.

Without being bound by theory, the inventors demonstrated stretchable and self-adhesive dry electrodes by using highly conductive blends of poly(ethylenedioxythiophene):poly (styrenesulfonate) (PEDOTSS, a conductive polymer), waterboie polyurethane (WPU, an elastomer) and D-sorbitol and investigate their application for epidermal biopotential monitoring. The blends can have high conductivity and stretchability arising from the bi-continuous networks formed by PEDOT:PSS and WPU, respectively. The presence of a sugar such as D-sorbitol enables the blends to have good adhesion on skin in both dry and wet states. The blend films can be used as dry electrodes for precise biopotential monitoring in various environments including dry/wet skin and during body motion. They can give rise to high-quality signals and be used for long-term biomedical monitoring. The signal quality is comparable to that with commercial gel electrodes which are not suitable for long-term biopotential monitoring. The benefits or advantages of the dry electrodes are as follow:

| Feature | Benefit/Advantage |
|---|---|
| Self-adhesive | The film material with good adhesion can attach tightly on smooth |
| | and hairy skin in both dry and wet state. Moreover, the adhesive film |
| | can stick stably on the deformed skin during body motion. |
| Biocompatibility | The material is made of completely biocompatible materials without |
| | any irradiation on the skin even after a long-term wearing. |
| Good Stretchablity | The film materials can be stretched larger than 40% without any |
| and mechanical | affection on the conductivity. The film with about 50 μm thickness |
| strength | can bear a weight larger than 0.25 KG, showing good mechanical |
| | properties for conveninent operation. |
| High quality of | Based on the high adhesion and conductivity, the film used as a |
| recording most of | biocompatible dry electrode can detect all the biosignal such as GSR, |
| biopotential | EMG, ECG, EEG with high signal quality and sensitivity. Taking the |
| | ECG measurement as an example, the film can record ECG on dry |
| | ans wet skin with little motion artifacts. The recorded ECG signal |
| | shows standard ECG pattern of PQRST waves without any tailing or |
| | distortion behaviors. |

The popular electrodes in clinic are made of Ag/AgCl gel electrolyte. Although the gel electrodes can give rise to high-quality signal, they are not suitable for wearable and long-term monitoring because of the evaporation of the liquid of the gel electrolyte. In an embodiment, the prepared PEDOT film dry electrode with competitive price shows comparable biosignal detection performance in the term of signal quality and sensitivity. More importantly, the PEDOT film electrode can be used more stably in a long-term without any reduction on the detection performance. Moreover, the prepared film dry electrode containing biocompatible materials is more friendly for the skin without any irradiation like commercial gel materials. In addition, the PEDOT film dry electrode can test the epidermal biosignal on the deformable skin in both dry and wet state, giving robust detection properties. So, the prepared PEDOT:PSS film shows immense potential of replacing present gel electrode, particularly for long-term healthcare monitoring.

The present invention provides a polymer composition comprising:
a) an electrically conductive polymer;
b) an elastomer; and
c) a sugar alcohol.

The polymer composition refers to a mixture of at least two entities, in which at least one of the entities is a polymer. When combined, a material with characteristics different from the individual components is produced. The composition can be formed as a liquid, or can be formed as a solid. For example, the polymer composition can further comprise a solvent, which can be an aqueous medium. When the polymer composition is cured, for example by heating to 60° C., a solid polymer composite can be formed.

In one embodiment, the polymeric composition is a dry polymeric composition.

As used herein, “electrically conductive polymer” or “conductive polymer” or “intrinsically conducting polymers” are organic polymers that conduct electricity. Such polymers may have metallic conductivity or can be semiconductors. Conductive polymers are generally not thermoplastics, i.e., they are not thermoformable. But, like insulating polymers, they are organic materials. They can offer high electrical conductivity but do not show similar mechanical properties to other commercially available polymers. Examples of electrically conductive polymers include, but are not limited to, polyacetylene, polyphenylene, polyphenylene vinylene, polypyrrole, polythiophene, polyaniline, polyphenylene sulphide, polycarbazole, polyindole, polyazepine, poly(fluorene)s, polypyrenes, polyazulenes, polynaphthalenes, and poly(3,4-ethylenedioxythiophene).

“Polymers” is a substance or material consisting of very large molecules, or macromolecules, composed of many repeating subunits. Polymers range from familiar synthetic plastics such as polystyrene to natural biopolymers such as DNA and proteins that are fundamental to biological structure and function. Polymers, both natural and synthetic, are created via polymerization of many small molecules, known as monomers. Their consequently large molecular mass, relative to small molecule compounds, produces unique physical properties including toughness, high elasticity, viscoelasticity, and a tendency to form amorphous and semicrystalline structures rather than crystals.

As used herein, “elastomer” is a polymer with viscoelasticity (i.e., both viscosity and elasticity) and has very weak intermolecular forces, generally low Young's modulus and high failure strain compared with other materials. It is a polymer that displays rubber-like elasticity. The term is often used interchangeably with rubber. Elastomers are amorphous polymers maintained above their glass transition temperature, so that considerable molecular reconformation, without breaking of covalent bonds, is feasible. At ambient temperatures, such rubbers are thus relatively compliant (E˜3 MPa) and deformable. Examples are, but not limited to, natural and synthetic polyisoprene, polybutadiene, chloroprene rubber, polychloroprene, Neoprene, Baypren, butyl rubber (copolymer of isobutene and isoprene), halogenated butyl rubbers (chloro butyl rubber; bromo butyl rubber), styrene-butadiene rubber (copolymer of styrene and butadiene), nitrile rubber (copolymer of butadiene and acrylonitrile), hydrogenated nitrile rubbers (HNBR), Therban, Zetpol, EPM (ethylene propylene rubber, a copolymer of ethene and propene), EPDM rubber (ethylene propylene diene rubber, a terpolymer of ethylene, propylene and a diene-component), epichlorohydrin rubber, polyacrylic rubber, silicone rubber, fluorosilicone rubber, fluoroelastomers, Viton, Tecnoflon, Fluorel, Aflas, Dai-El, perfluoroelastomers, Tecnoflon PFR, Kalrez, Chemraz, Perlast, polyether block amides, chlorosulfonated polyethylene, and ethylene-vinyl acetate. Elastomers also includes thermoplastic elastomers, proteins resilin and elastin, polysulfide rubber and elastolefin.

Thermoplastic elastomers, sometimes referred to as thermoplastic rubbers, are a class of copolymers or a physical mix of polymers (usually a plastic and a rubber) that consist of materials with both thermoplastic and elastomeric properties. Thermoplastic elastomers show advantages typical of both rubbery materials and plastic materials. The benefit of using thermoplastic elastomers is the ability to stretch to moderate elongations and return to its near original shape creating a longer life and better physical range than other materials. The principal difference between thermoset elastomers and thermoplastic elastomers is the type of cross-linking bond in their structures. In fact, crosslinking is a critical structural factor which imparts high elastic properties. A thermoplastic elastomer typically has three characteristics: the ability to be stretched to moderate elongations and, upon the removal of stress, return to something close to its original shape; processable as a melt at elevated temperature; and absence of significant creep.

Examples of thermoplastic elastomers (designations according to ISO 18064) are styrenic block copolymers, TPS (TPE-s); thermoplastic polyolefinelastomers, TPO (TPE-o); thermoplastic Vulcanizates, TPV (TPE-v or TPV); thermoplastic polyurethanes, TPU (TPU); thermoplastic copolyester, TPC (TPE-E); thermoplastic polyamides, TPA (TPE-A); not classified thermoplastic elastomers, TPZ. Examples of TPE materials that come from block copolymers group are amongst others CAWITON, THERMOLAST K, THERMOLAST M, Arnitel, Hytrel, Dryflex, Mediprene, Kraton, Pibiflex, Sofprene, and Laprene. Out of these styrenic block copolymers (TPE-s) are CAWITON, THERMOLAST K, THERMOLAST M, Sofprene, Dryflex and Laprene. Laripur, Desmopan or Elastollan are examples of thermoplastic polyurethanes (TPU). Sarlink, Santoprene, Termoton, Solprene, THERMOLAST V, Vegaprene, or Forprene are examples of TPV materials. Examples of thermoplastic olefin elastomers (TPO) compound are For-Tec E or Engage, or Ninjaflex.

As used herein, “sugar alcohols” (also called polyhydric alcohols, polyalcohols, alditols or glycitols) are organic compounds, typically derived from sugars, containing at least two hydroxyl group (—OH) which are attached to carbon atoms. They are water-soluble solids that can occur naturally or be produced industrially by hydrogenation of sugars. Since they contain multiple —OH groups, they can also be classified as polyols. They can be monosaccharides, disaccharides or polysaccharides. Examples of sugar alcohols are ethylene glycol (2-carbon), glycerol (3-carbon), erythritol (4-carbon), threitol (4-carbon), arabitol (5-carbon), xylitol (5-carbon), ribitol (5-carbon), mannitol (6-carbon), sorbitol (6-carbon), galactitol (6-carbon), fucitol (6-carbon), iditol (6-carbon), inositol (6-carbon; a cyclic sugar alcohol), volemitol (7-carbon), isomalt (12-carbon), maltitol (12-carbon), lactitol (12-carbon), maltotriitol (18-carbon), maltotetraitol (24-carbon), and polyglycitol.

It was found that the addition of sugar alcohol can improve the conductivity and the stretchability of the polymer composite. In this regard, the sugar alcohol can act as a plasticiser.

The present invention provides a polymer composite comprising:
a) an electrically conductive polymer;
b) an elastomer; and
c) a sugar alcohol.

As used herein, ‘composite’ is a material made from two or more constituent materials with different physical or chemical properties that, when combined, produce a material with characteristics different from the individual components. The composite is formed as a solid.

In some embodiments, the polymer composite comprising:
a) an electrically conductive polymer comprising a polythiopine polymer and a polymeric acid dopant;
b) a elastomer; and
c) a sugar alcohol.

In some embodiments, the electrically conductive polymer comprises poly(ethylenedioxythiophene):poly(styrenesulfonate) (PEDOT:PSS).

In some embodiments, the PEDOT:PSS is at a ratio of about 2.5:1 w/w. In other embodiments, the PEDOT:PSS is at a ratio of about 4:1 w/w to about 1.5:1 w/w, about 4:1 w/w to about 2:1 w/w, or about 3.5:1 w/w to about 2:1 w/w, or about 3:1 w/w to about 2:1 w/w. In other embodiments, the PEDOT:PSS is at a ratio of about 1.5:1 w/w, about 2:1 w/w, about 3:1 w/w, about 3.5:1 w/w, or about 4:1 w/w.

In some embodiments, the electrically conductive polymer is about 4 wt % to about 30 wt % of polymer composite. In other embodiments, it is about 4 wt % to about 25 wt %. In other embodiments, it is about 8 wt % to about 25 wt %, about 12 wt % to about 25 wt %, about 15 wt % to about 25 wt %, or about 15 wt % to about 20 wt %. In other embodiments, it is about 8 wt %, about 10 wt %, about 12 wt %, about 14 wt %, about 16 wt %, about 18 wt %, about 19 wt %, about 20 wt %, about 22 wt %, about 24 wt %, or about 25 wt %.

In some embodiments, the polymeric composite comprising:
a) an electrically conductive polymer;
b) a thermoplastic elastomer; and
c) a sugar alcohol.

In some embodiments, the elastomer is selected from the group consisting of styrenic block copolymers, thermoplastic polyolefinelastomers, thermoplastic Vulcanizates, thermoplastic polyurethanes, thermoplastic copolyester, thermoplastic polyamides, or a combination thereof. In other embodiments, the elastomer is a waterborne polyurethane (WPU). Polyurethane (PU) is a polymer composed of organic units joined by carbamate (urethane) links.

The waterborne polyurethane or polyurethane dispersion is understood to be a polyurethane polymer resin that is dispersible in an aqueous medium. Its manufacture involves the synthesis of polyurethanes having carboxylic acid functionality or nonionic hydrophiles like PEG incorporated into, or pendant from, the polymer backbone. The presence of hydrophilic groups can allow the polymer composite to be favourable adhered to a skin surface. Additionally, WPU can act as an elastomer to get a stretchable composite.

For example, the WPU can be Aqua ZAR Polyurethane, a water-borne paint coating purchasable from ZAR. In some embodiments, the WPU further comprises dipropylene glycol monomethyl ether, 1-(2-butoxy-1-methylethoxy)-2-propanol, amorphous silica, or a combination thereof. Dipropylene glycol monomethyl ether can be about 5 wt % to about 10 wt % of WPU. 1-(2-butoxy-1-methylethoxy)-2-propanol can be about 1 wt % to about 5 wt % of WPU. Amorphous silica can be about 1 wt % to about 5 wt % of WPU.

In some embodiments, the WPU is about 37 wt % to 58 wt % of the polymer composite. In other embodiments, WPU is about 37 wt % to 55 wt %, about 37 wt % to 50 wt %, about 37 wt % to 45 wt %, about 37 wt % to 43 wt %, or about 37 wt % to 40 wt %. In other embodiments, WPU is about 37 wt %, about 40 wt %, about 43 wt %, about 45 wt %, about 50 wt %, about 55 wt %, or about 58 wt %.

In some embodiments, the sugar alcohol is selected from the group consisting of ethylene glycol, glycerol, erythritol, threitol, arabitol, xylitol, ribitol, mannitol, sorbitol, galactitol, fucitol, iditol, inositol, volemitol, isomalt, maltitol, lactitol, maltotriitol, maltotetraitol, polyglycitol, or a combination thereof. In other embodiments, the sugar alcohol is sorbitol. In other embodiments, the sugar alcohol is D-sorbitol. In other embodiments, the sugar alcohol is D-sorbitol and ethylene glycol.

In some embodiments, the sugar alcohol is about 15 wt % to about 50 wt % of polymer composite. In other embodiments, it is about 20 wt % to about 50 wt %, about 20 wt % to about 45 wt %, about 20 wt % to about 40 wt %, about 25 wt % to about 50 wt % about 25 wt % to about 45 wt %, about 30 wt % to about 40 wt %, or about 35 wt % to about 40 wt %. In other embodiments, it is about 38 wt %. In some preferred embodiments, it is about 20 wt % to about 40 wt %, or about 30 wt % to about 40 wt %.

The addition of sugar alcohol into the polymer composite acts to synergistically improve the adhesion and stretchability properties of the polymer composite. It is believed that this is due to the hydroxyl groups which interacts with the electrically conductive polymer and/or the elastomer. For example, the sugar alcohol can serve as a plasticizer for PEDOT:PSS. It can improve the conductivity and the stretchability. The mechanism for the stretchability improvement by sugar alcohol is ascribed to the softening of electrically conductive polymer and/or the elastomer chains. Sugar alcohol can position among the electrically conductive polymer chains and thus destructs the interaction among the electrically conductive polymer chains. This makes the conformational change of the electrically conductive polymer chains under stress become easy and thus increases the mechanical flexibility of PEDOT:PSS.

In some embodiments, the polymer composite comprises PEDOT:PSS, waterborne polyurethane (WPU) and D-sorbitol. The chemical structures of these components are shown below:

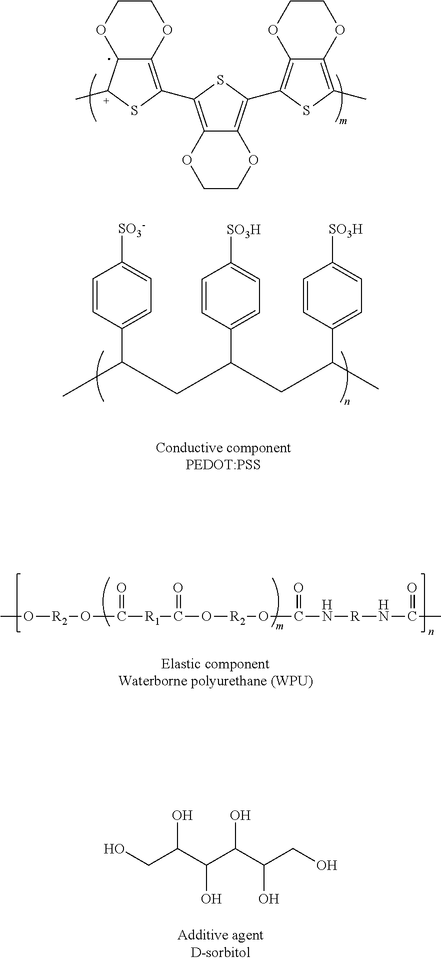

Accordingly, the present invention provides a polymer composite, comprising:
a) poly(ethylenedioxythiophene):poly(styrenesulfonate) (PEDOT:PSS);
b) waterborne polyurethane (WPU); and
c) D-sorbitol.

In some embodiments, the polymer composite comprises:
a) poly(ethylenedioxythiophene):poly(styrenesulfonate) (PEDOT:PSS) at a ratio of about 2.5:1 w/w;
b) waterborne polyurethane (WPU); and
c) D-sorbitol.

In some embodiments, the polymer composite comprises:
a) poly(ethylenedioxythiophene):poly(styrenesulfonate) (PEDOT:PSS) at a ratio of about 2.5:1 w/w;
b) waterborne polyurethane (WPU); and
c) D-sorbitol;
wherein (PEDOT:PSS) is about 4 wt % to about 25 wt % of polymer composite.

In some embodiments, the polymer composite comprises:
a) poly(ethylenedioxythiophene):poly(styrenesulfonate) (PEDOT:PSS) at a ratio of about 2.5:1 w/w;
b) waterborne polyurethane (WPU); and
c) D-sorbitol;
wherein (PEDOT:PSS) is about 19 wt % of polymer composite.

In some embodiments, the polymer composite comprises:
a) poly(ethylenedioxythiophene):poly(styrenesulfonate) (PEDOT:PSS) at a ratio of about 2.5:1 w/w;
b) waterborne polyurethane (WPU); and
c) D-sorbitol;
   wherein (PEDOT:PSS) is about 4 wt % to about 25 wt % of polymer composite; and
   wherein WPU is about 37 wt % to 58 wt % of polymer composite.

In some embodiments, the polymer composite comprises:
a) poly(ethylenedioxythiophene):poly(styrenesulfonate) (PEDOT:PSS) at a ratio of about 2.5:1 w/w;
b) waterborne polyurethane (WPU); and
c) D-sorbitol;
   wherein (PEDOT:PSS) is about 4 wt % to about 25 wt % of polymer composite;
   wherein WPU is about 37 wt % to 58 wt % of polymer composite; and
   wherein sugar alcohol is about 38 wt % of polymer composite.

In some embodiments, the polymer composite further comprises ethylene glycol. Ethylene glycol can be added at about 0.2 wt % to 1.2 wt % of polymer composite. In other embodiments, ethylene glycol is added at about 0.2 wt % to 1.1 wt %, about 0.2 wt % to 1 wt %, about 0.2 wt % to 0.9 wt %, about 0.2 wt % to 0.8 wt %, about 0.2 wt % to 0.7 wt %, about 0.2 wt % to 0.6 wt %, or about 0.2 wt % to 0.5 wt %.

The addition of ethylene glycol (an additional sugar alcohol) provides a further advantage in that it can further increase the conductivity of the polymer composites.

Further advantageously, it was found that to form the polymer composite, a further curing agent and/or surfactant is not required. This allows the polymer composite to have a low toxicity, as commonly used curing agents and/or surfactants can be toxic and harmful to people. Further, such molecules can also leach out from the polymer composites, thus providing a long term toxicology issue. In some embodiments, the polymer composite does not include surfactant. Surfactants are compounds that lower the surface tension (or interfacial tension) between two liquids, between a gas and a liquid, or between a liquid and a solid.

Surfactants are amphiphilic molecules that have hydrophobic and hydrophilic parts, and can be cationic, anionic, non-ionic or zwitterionic.

Accordingly, in one aspect, the polymer composite consist essentially of:
a) an electrically conductive polymer;
b) a elastomer; and
c) a sugar alcohol.

The term “consisting essentially of” is construed to include the specified materials or steps, as well as other materials or steps that do not materially affect the working of the claimed invention.

In one aspect, the polymer composite consist essentially of:
a) an electrically conductive polymer;
b) a elastomer;
c) a d-sorbitol; and
d) ethylene glycol.

In one aspect, the polymer composite consist essentially of:
a) poly(ethylenedioxythiophene):poly(styrenesulfonate) (PEDOT:PSS);
b) waterborne polyurethane (WPU);
c) D-sorbitol; and
d) Ethylene glycol.

In one aspect, the polymer composite consist essentially of:
a) poly(ethylenedioxythiophene):poly(styrenesulfonate) (PEDOT:PSS) at a ratio of about 2.5:1 w/w;
b) waterborne polyurethane (WPU);
c) D-sorbitol; and
d) Ethylene glycol.

Accordingly, in one aspect, the polymer composite consist of:
a) an electrically conductive polymer;
b) a elastomer; and
c) a sugar alcohol.

The term “consisting of” is generally interpreted to be closed ended—the feature will be selected only from the listed alternatives. Thus, “a combination consisting of components A and B” would not include a combination of components A, B and C.

In one aspect, the polymer composite consist of:
a) an electrically conductive polymer;
b) a elastomer;
c) a d-sorbitol; and
d) ethylene glycol.

In one aspect, the polymer composite consist of:
a) poly(ethylenedioxythiophene):poly(styrenesulfonate) (PEDOT:PSS);
b) waterborne polyurethane (WPU);
c) D-sorbitol; and
d) Ethylene glycol.

In one aspect, the polymer composite consist of:
a) poly(ethylenedioxythiophene):poly(styrenesulfonate) (PEDOT:PSS) at a ratio of about 2.5:1 w/w;
b) waterborne polyurethane (WPU);
c) D-sorbitol; and
d) Ethylene glycol.

In some embodiments, a surface of the polymer composite has a nanoscale grainy morphology of about 50 nm to about 150 nm. In other embodiments, the grainy morphology is about 60 nm to about 140 nm, about 70 nm to about 130 nm, about 80 nm to about 120 nm, or about 90 nm to about 110 nm. In other embodiments, the grainy morphology is about 100 nm.

In some embodiments, the polymer composite has a surface roughness of about 10 nm to about 20 nm. In other embodiments, the surface roughness is about 12 nm, about 14 nm, about 16 nm, or about 18 nm.

The polymer composite can be provided as a homogenous blend of entities. In some embodiments, the electrically conductive polymer and elastomer each form separate continuous phases in the polymer composite.

The polymer composite is stretchable. In some embodiments, when PEDOT:PSS loading is about 4 wt % of the polymer composite, the elongation at break is about 200%. In some embodiments, when PEDOT:PSS loading is about 19 wt % of the polymer composite, the elongation at break is about 35% to about 50%. In other embodiments, the elongation at break is about 37%, about 39%, about 41%, about 43%, about 45%, about 47% or about 49%.

In some embodiments, when PEDOT:PSS loading is about 4 wt % of the polymer composite, the Young's modulus is about 2 MPa. In some embodiments, when PEDOT:PSS loading is about 19 wt % of the polymer composite, the Young's modulus is about 80 MPa to about 90 MPa. In other embodiments, the Young's modulus is about 82 MPa to about 90 MPa, about 82 MPa to about 88 MPa, about 82 MPa to about 86 MPa, or about 84 MPa to about 86 MPa.

In some embodiments, the conductivity of the polymer composite is about 60 S/cm to about 600 S/cm. This can be when the PEDOT:PSS loading in the polymer composite is increased from 4 wt % to 25 wt %. The conductivity can be linearly correlated to the PEDOT:PSS loading. In other embodiments, conductivity is about 60 S/cm to about 590 S/cm, about 60 S/cm to about 580 S/cm, about 60 S/cm to about 570 S/cm, about 60 S/cm to about 560 S/cm, about 60 S/cm to about 550 S/cm, about 65 S/cm to about 590 S/cm, or about 70 S/cm to about 590 S/cm. In other embodiments, the conductivity of the polymer composite is about 72 S/cm to about 545 S/cm.

In some embodiments, the polymer composite is repeatedly stretchable for at least 400 cycles. In other embodiments, the polymer composite is repeatedly stretchable for at least 300 cycles, at least 200 cycles or at least 100 cycles. When the polymer composite is repeatedly stretched, a variation of conductance is less than about 10%. In other embodiments, the variation is less than about 9%, about 8%, about 7%, about 6%, about 5%, or about 4%.

In some embodiments, when the strain range is about 30%, a resistance variation is less than about 7%. In other embodiments, the resistance variation is less than about 6%, 5.5%, 5%, 4.5%, 4%, 3.5%, 3%, or 2.5%.

In some embodiments, the polymer composite has an adhesion force to a skin of about 0.35 N/cm to about 0.7 N/cm. The skin can be a sample of porcine skin or human skin. The skin can also have a surface which is dry or wet. In other embodiments, the adhesion force is about 0.4 N/cm to about 0.7 N/cm, about 0.4 N/cm to about 0.65 N/cm, about 0.4 N/cm to about 0.6 N/cm, about 0.45 N/cm to about 0.6 N/cm, or about 0.45 N/cm to about 0.55 N/cm. In other embodiments, the adhesion force to a dry skin is about 0.43 N/cm. In other embodiments, the adhesion force to a dry skin is about 0.55 N/cm. In other embodiments, the adhesion force to a wet skin is about 0.56 N/cm.

In some embodiments, the polymer composite has an adhesion force to a glass surface of about 1 N/cm to about 3 N/cm. In other embodiments, the adhesion force is about 1 N/cm to about 2.9 N/cm, about 1.1 N/cm to about 2.9 N/cm, about 1.1 N/cm to about 2.8 N/cm, about 1.1 N/cm to about 2.7 N/cm, about 1.2 N/cm to about 2.7 N/cm, about 1.3 N/cm to about 2.7 N/cm, about 1.3 N/cm to about 2.6 N/cm, about 1.4 N/cm to about 2.6 N/cm, or about 1.4 N/cm to about 2.5 N/cm. In other embodiments, the adhesion force is about 1.2 N/cm, about 1.3 N/cm, about 1.4 N/cm, about 1.44 N/cm, about 1.5 N/cm, about 1.6 N/cm, about 1.7 N/cm, about 1.8 N/cm, about 1.9 N/cm, about 2 N/cm, about 2.1 N/cm, about 2.2 N/cm, about 2.3 N/cm, about 2.4 N/cm, about 2.5 N/cm, about 2.6 N/cm, about 2.7 N/cm, about 2.8 N/cm, about 2.9 N/cm, or about 3 N/cm.

In some embodiments, when the polymer composite is stretched to a strain of 30%, the adhesion force to a skin is about 0.46 N/cm. The adhesion force does not vary substantially when the polymer is in a resting state or in a stretched state. In some embodiments, the variation in the adhesion force between a resting state and a stretched state is less than about 10%, about 9%, about 8%, about 7%, about 6%, or about 5%.

In some embodiments, the polymer composite has a thickness of about 10 μm to about 30 μm. In other embodiments, the thickness is about 12 μm to about 30 μm, about 14 μm to about 30 μm, about 14 μm to about 28 μm, about 14 μm to about 26 μm, about 14 μm to about 24 μm, about 14 μm to about 22 μm, about 16 μm to about 22 μm, or about 18 μm to about 22 μm. In other embodiments, the thickness is about 12 μm, about 14 μm, about 16 μm, about 18 μm, about 20 μm, about 22 μm, about 24 μm, about 26 μm, about 28 μm, or about 30 μm.

When the polymer composite having a thickness of about 20 μm is stretched, the thickness can decrease to about 15 μm. In some embodiments, the polymer composite has a stretchability of about 30% to about 60%, about 30% to about 55%, about 30% to about 50%, about 35% to about 50%, or about 35% to about 45%. In some embodiments, the stretchability is more than about 30%, about 35%, about 40%, about 45%, about 50%, about 55%, or about 60%.

In one aspect, the polymer composite comprises
a) poly(ethylenedioxythiophene):poly(styrenesulfonate) (PEDOT:PSS);
b) waterborne polyurethane (WPU); and
c) a sugar alcohol;
   wherein a ratio of PEDOT to PSS is about 2:1 w/w to about 3:1 w/w.

In one aspect, the polymer composite comprises
a) poly(ethylenedioxythiophene):poly(styrenesulfonate) (PEDOT:PSS);
b) waterborne polyurethane (WPU); and
c) a d-sorbitol;
   wherein a ratio of PEDOT to PSS is about 2:1 w/w to about 3:1 w/w.

In one aspect, the polymer composite comprises
a) poly(ethylenedioxythiophene):poly(styrenesulfonate) (PEDOT:PSS);
b) waterborne polyurethane (WPU); and
c) a sugar alcohol;
   wherein a ratio of PEDOT to PSS is about 2:1 w/w to about 3:1 w/w; and
   wherein PEDOT:PSS is about 4 wt % to about 25 wt % of the polymer composite.

In one aspect, the polymer composite comprises
a) poly(ethylenedioxythiophene):poly(styrenesulfonate) (PEDOT:PSS);
b) waterborne polyurethane (WPU); and
c) a d-sorbitol;
   wherein a ratio of PEDOT to PSS is about 2:1 w/w to about 3:1 w/w; and
   wherein PEDOT:PSS is about 4 wt % to about 25 wt % of the polymer composite.

In one aspect, the polymer composite comprises
a) poly(ethylenedioxythiophene):poly(styrenesulfonate) (PEDOT:PSS);
b) waterborne polyurethane (WPU); and
c) a sugar alcohol;
   wherein a ratio of PEDOT to PSS is about 2.5:1 w/w; and
   wherein PEDOT:PSS is about 4 wt % to about 25 wt % of the polymer composite.

In one aspect, the polymer composite comprises
a) poly(ethylenedioxythiophene):poly(styrenesulfonate) (PEDOT:PSS);
b) waterborne polyurethane (WPU); and
c) a d-sorbitol;
   wherein a ratio of PEDOT to PSS is about 2.5:1 w/w; and
   wherein PEDOT:PSS is about 4 wt % to about 25 wt % of the polymer composite.

In one aspect, the polymer composite comprises
a) poly(ethylenedioxythiophene):poly(styrenesulfonate) (PEDOT:PSS);
b) waterborne polyurethane (WPU); and
c) a sugar alcohol;
   wherein a ratio of PEDOT to PSS is about 2.5:1 w/w;
   wherein PEDOT:PSS is about 4 wt % to about 25 wt % of the polymer composite;
   wherein when PEDOT:PSS is about 4 wt % of the polymer composite, the polymer composite has a elongation at break of about 200%.

In one aspect, the polymer composite comprises
a) poly(ethylenedioxythiophene):poly(styrenesulfonate) (PEDOT:PSS);
b) waterborne polyurethane (WPU); and
c) a sugar alcohol;
   wherein a ratio of PEDOT to PSS is about 2.5:1 w/w;
   wherein PEDOT:PSS is about 4 wt % to about 25 wt % of the polymer composite;
   wherein when PEDOT:PSS is about 4 wt % of the polymer composite, the polymer composite has a elongation at break of about 200%; and
   wherein when PEDOT:PSS is about 19 wt % of the polymer composite, the polymer composite has a elongation at break of about 40%.

In one aspect, the polymer composite comprises
a) poly(ethylenedioxythiophene):poly(styrenesulfonate) (PEDOT:PSS);
b) waterborne polyurethane (WPU); and
c) a d-sorbitol;
   wherein a ratio of PEDOT to PSS is about 2.5:1 w/w;
   wherein PEDOT:PSS is about 4 wt % to about 25 wt % of the polymer composite;
   wherein when PEDOT:PSS is about 4 wt % of the polymer composite, the polymer composite has a elongation at break of about 200%; and
   wherein when PEDOT:PSS is about 19 wt % of the polymer composite, the polymer composite has a elongation at break of about 40%.

In one aspect, the polymer composite comprises
a) poly(ethylenedioxythiophene):poly(styrenesulfonate) (PEDOT:PSS);
b) waterborne polyurethane (WPU); and
c) a sugar alcohol;
   wherein a ratio of PEDOT to PSS is about 2.5:1 w/w;
   wherein PEDOT:PSS is about 4 wt % to about 25 wt % of the polymer composite; and
   wherein when PEDOT:PSS is about 4 wt % of the polymer composite, the polymer composite has a Young's modulus of about 2 MPa.

In one aspect, the polymer composite comprises
a) poly(ethylenedioxythiophene):poly(styrenesulfonate) (PEDOT:PSS);
b) waterborne polyurethane (WPU); and
c) a sugar alcohol;
   wherein a ratio of PEDOT to PSS is about 2.5:1 w/w;
   wherein PEDOT:PSS is about 4 wt % to about 25 wt % of the polymer composite;
   wherein when PEDOT:PSS is about 4 wt % of the polymer composite, the polymer composite has a Young's modulus of about 2 MPa; and
   wherein when PEDOT:PSS is about 19 wt % of the polymer composite, the polymer composite has a Young's modulus of about 85 MPa.

In one aspect, the polymer composite comprises
a) poly(ethylenedioxythiophene):poly(styrenesulfonate) (PEDOT:PSS);
b) waterborne polyurethane (WPU); and
c) a d-sorbitol;
   wherein a ratio of PEDOT to PSS is about 2.5:1 w/w;
   wherein PEDOT:PSS is about 4 wt % to about 25 wt % of the polymer composite;
   wherein when PEDOT:PSS is about 4 wt % of the polymer composite, the polymer composite has a Young's modulus of about 2 MPa; and
   wherein when PEDOT:PSS is about 19 wt % of the polymer composite, the polymer composite has a Young's modulus of about 85 MPa.

In one aspect, the polymer composite comprises
a) poly(ethylenedioxythiophene):poly(styrenesulfonate) (PEDOT:PSS);
b) waterborne polyurethane (WPU); and
c) a sugar alcohol;
   wherein a ratio of PEDOT to PSS is about 2.5:1 w/w; and
   wherein PEDOT:PSS is about 19 wt % of the polymer composite.

In one aspect, the polymer composite comprises
a) poly(ethylenedioxythiophene):poly(styrenesulfonate) (PEDOT:PSS);
b) waterborne polyurethane (WPU); and
c) a d-sorbitol;
   wherein a ratio of PEDOT to PSS is about 2.5:1 w/w;
   wherein PEDOT:PSS is about 19 wt % of the polymer composite.

In one aspect, the polymer composite comprises
a) poly(ethylenedioxythiophene):poly(styrenesulfonate) (PEDOT:PSS);
b) waterborne polyurethane (WPU);
c) a d-sorbitol; and
d) ethylene glycol;
   wherein a ratio of PEDOT to PSS is about 2.5:1 w/w;
   wherein PEDOT:PSS is about 19 wt % of the polymer composite.

In one aspect, the polymer composite comprises
a) poly(ethylenedioxythiophene):poly(styrenesulfonate) (PEDOT:PSS);
b) waterborne polyurethane (WPU);
c) a d-sorbitol; and
d) ethylene glycol;
   wherein a ratio of PEDOT to PSS is about 2.5:1 w/w;
   wherein PEDOT:PSS is about 19 wt % of the polymer composite.
   wherein ethylene glycol is about 0.2 wt % to 1.2 wt % of the polymer composite.

In one aspect, the polymer composite comprises
a) poly(ethylenedioxythiophene):poly(styrenesulfonate) (PEDOT:PSS);
b) waterborne polyurethane (WPU); and
c) a sugar alcohol;
   wherein the sugar alcohol is about 20 wt % to about 50 wt % of the polymer composite.

In one aspect, the polymer composite comprises
a) poly(ethylenedioxythiophene):poly(styrenesulfonate) (PEDOT:PSS);
b) waterborne polyurethane (WPU); and
c) a d-sorbitol;
   wherein the d-sorbitol is about 20 wt % to about 50 wt % of the polymer composite.

In one aspect, the polymer composite comprises
a) poly(ethylenedioxythiophene):poly(styrenesulfonate) (PEDOT:PSS);
b) waterborne polyurethane (WPU); and
c) a sugar alcohol;
   wherein the sugar alcohol is about 20 wt % to about 50 wt % of the polymer composite; and
   wherein a ratio of PEDOT to PSS is about 2:1 w/w to about 3:1 w/w.

In one aspect, the polymer composite comprises
a) poly(ethylenedioxythiophene):poly(styrenesulfonate) (PEDOT:PSS);
b) waterborne polyurethane (WPU); and
c) a d-sorbitol;
   wherein the d-sorbitol is about 20 wt % to about 50 wt % of the polymer composite; and
   wherein a ratio of PEDOT to PSS is about 2:1 w/w to about 3:1 w/w.

In one aspect, the polymer composite comprises
a) poly(ethylenedioxythiophene):poly(styrenesulfonate) (PEDOT:PSS);
b) waterborne polyurethane (WPU); and
c) a sugar alcohol;
   wherein the sugar alcohol is about 20 wt % to about 50 wt % of the polymer composite;
   wherein a ratio of PEDOT to PSS is about 2:1 w/w to about 3:1 w/w; and
   wherein PEDOT:PSS is about 4 wt % to about 25 wt % of the polymer composite.

In one aspect, the polymer composite comprises
a) poly(ethylenedioxythiophene):poly(styrenesulfonate) (PEDOT:PSS);
b) waterborne polyurethane (WPU); and
c) a d-sorbitol;
   wherein the d-sorbitol is about 20 wt % to about 50 wt % of the polymer composite;
   wherein a ratio of PEDOT to PSS is about 2:1 w/w to about 3:1 w/w; and
   wherein PEDOT:PSS is about 4 wt % to about 25 wt % of the polymer composite.

In one aspect, the polymer composite comprises
a) poly(ethylenedioxythiophene):poly(styrenesulfonate) (PEDOT:PSS);
b) waterborne polyurethane (WPU);
c) a sugar alcohol; and
d) ethylene glycol;
   wherein the sugar alcohol is about 20 wt % to about 50 wt % of the polymer composite;
   wherein a ratio of PEDOT to PSS is about 2:1 w/w to about 3:1 w/w; and
   wherein PEDOT:PSS is about 4 wt % to about 25 wt % of the polymer composite.

In one aspect, the polymer composite comprises
a) poly(ethylenedioxythiophene):poly(styrenesulfonate) (PEDOT:PSS);
b) waterborne polyurethane (WPU);
c) a d-sorbitol; and
d) ethylene glycol;
   wherein the d-sorbitol is about 20 wt % to about 50 wt % of the polymer composite;
   wherein a ratio of PEDOT to PSS is about 2:1 w/w to about 3:1 w/w; and
   wherein PEDOT:PSS is about 4 wt % to about 25 wt % of the polymer composite.

In one aspect, the polymer composite comprises
a) poly(ethylenedioxythiophene):poly(styrenesulfonate) (PEDOT:PSS);
b) waterborne polyurethane (WPU);
c) a sugar alcohol; and
d) ethylene glycol;
   wherein the sugar alcohol is about 20 wt % to about 50 wt % of the polymer composite;
   wherein a ratio of PEDOT to PSS is about 2:1 w/w to about 3:1 w/w; and
   wherein PEDOT:PSS is about 4 wt % to about 25 wt % of the polymer composite; and
   wherein ethylene glycol is about 0.2 wt % to 1.2 wt % of the polymer composite.

In one aspect, the polymer composite comprises
a) poly(ethylenedioxythiophene):poly(styrenesulfonate) (PEDOT:PSS);
b) waterborne polyurethane (WPU);
c) a d-sorbitol; and
d) ethylene glycol;
   wherein the d-sorbitol is about 20 wt % to about 50 wt % of the polymer composite;
   wherein a ratio of PEDOT to PSS is about 2:1 w/w to about 3:1 w/w; and
   wherein PEDOT:PSS is about 4 wt % to about 25 wt % of the polymer composite; and
   wherein ethylene glycol is about 0.2 wt % to 1.2 wt % of the polymer composite.

In one aspect, the polymer composite comprises
a) poly(ethylenedioxythiophene):poly(styrenesulfonate) (PEDOT:PSS);
b) waterborne polyurethane (WPU);
c) a d-sorbitol; and
d) ethylene glycol;
   wherein the d-sorbitol is about 38 wt % of the polymer composite;
   wherein a ratio of PEDOT to PSS is about 2:1 w/w to about 3:1 w/w; and
   wherein PEDOT:PSS is about 4 wt % to about 25 wt % of the polymer composite; and
   wherein ethylene glycol is about 0.2 wt % to 1.2 wt % of the polymer composite.

In one aspect, the polymer composite comprises
a) poly(ethylenedioxythiophene):poly(styrenesulfonate) (PEDOT:PSS);
b) waterborne polyurethane (WPU);
c) a d-sorbitol; and
d) ethylene glycol;
   wherein the d-sorbitol is about 38 wt % of the polymer composite;
   wherein a ratio of PEDOT to PSS is about 2.5:1 w/w; and
   wherein PEDOT:PSS is about 4 wt % to about 25 wt % of the polymer composite; and
   wherein ethylene glycol is about 0.2 wt % to 1.2 wt % of the polymer composite.

In one aspect, the polymer composite comprises
a) poly(ethylenedioxythiophene):poly(styrenesulfonate) (PEDOT:PSS);
b) waterborne polyurethane (WPU);
c) a d-sorbitol; and
d) ethylene glycol;
   wherein the d-sorbitol is about 38 wt % of the polymer composite;
   wherein a ratio of PEDOT to PSS is about 2.5:1 w/w; and
   wherein PEDOT:PSS is about 19 wt % of the polymer composite; and
   wherein ethylene glycol is about 0.2 wt % to 1.2 wt % of the polymer composite.

Also disclosed herein is an electrical device comprising a conductive polymer composition and/or polymer composite as disclosed herein. As is shown herein, the electrode formed from the polymer composite is particularly advantageous for adhesion to skin and glass.

In some embodiments, the electrical device is an electrode. In other embodiments, the electrical device is a dry contact electrode. In other embodiments, the electrical device is a wearable device or electrode.

When used as an electrode, the electrode can have an electrode-skin electrical impedance at 10 Hz of about 70 KΩ cm² to about 100 KΩ cm². In other embodiments, the impedance is about 70 KΩ cm² to about 95 KΩ cm², about 70 KΩ cm² to about 90 KΩ cm², about 70 KΩ cm² to about 85 KΩ cm², about 75 KΩ cm² to about 85 KΩ cm², or about 80 KΩ cm² to about 85 KΩ cm². In other embodiments, the impedance is about 70 KΩ cm², about 75 KΩ cm², about 80 KΩ cm², about 85 KΩ cm², about 90 KΩ cm², about 95 KΩ cm², or about 100 KΩ cm².

The polymer composite can be used as a dry contact electrode for biopotential detection. In some embodiments, the polymer composite can be used for epidermal biopotential detection. For example, electrocardiogram (ECG) signals can be detected using the polymer composite as electrodes. In some embodiments, an ECG peak-to-peak voltage is about 1.6 mV to about 2 mV can be obtained. In other embodiments, ECG peak-to-peak voltage is about 1.65 mV to about 2 mV, about 1.7 mV to about 2 mV, about 1.7 mV to about 1.95 mV, about 1.7 mV to about 1.9 mV, about 1.75 mV to about 1.9 mV, about 1.8 mV to about 1.9 mV, or about 1.8 mV to about 1.85 mV. In some embodiments, an ECG peak-to-peak voltage is about 1.6 mV, about 1.65 mV, about 1.7 mV, about 1.75 mV, about 1.8 mV, about 1.85 mV, about 1.9 mV, about 1.95 mV, or about 2 mV.

In some embodiments, when an electrocardiogram (ECG) pulse in a frequency range of about 0 to 45 Hz is Fourier transformed, PQRST peaks is distinguishable along with the power of the signal in 20-40 dB.

In some embodiments, the ECG signal has a root-mean-square (RMS) noise of less than about 28 μV. In other embodiments, the RMS noise is less than about 27 μV, about 26 μV, about 25 μV, about 24 μV, about 23 μV, about 22 μV, about 21 μV, or about 20 V.

In some embodiments, when the electrode is subjected to movement or vibration, the ECG signal has a root-mean-square (RMS) noise of less than about 45 μV. In other embodiments, the RMS noise is less than about 44 μV, about 43 μV, about 42 μV, about 41 μV, about 40 μV, about 39 μV, about 38 μV, or about 37 V.

In some embodiments, the electrode can detect atrial fibrillation in a subject. This is done by identifying electrocardiographic arrhythmia. Atrial fibrillation is an abnormal heart rhythm (arrhythmia) characterized by the rapid and irregular beating of the atrial chambers of the heart. It often begins as short periods of abnormal beating, which become longer or continuous over time. The electrode can detect brief but significant increase in muscle contraction during tendon hyper-flexion testing and sustained the increase in muscle contraction against resistance before normalizing during relaxation.

The polymer composite can be used as a dry contact electrode for detecting an action potential generated by muscle fibers. In this application, an electromyogram (EMG) signal can be generated using the electrode. The peak-to-peak amplitude and the signal intensity are consistent with the applied gripping force. In some embodiments, a peak-to-peak amplitude is linearly correlated to an applied force. In other embodiments, a signal intensity is linearly correlated to an applied force. In some embodiments, the EMG signal is about 1 KHz to about 30 KHz, or about 1 KHz to about 20 KHz.

In some embodiments, EMG signals generated from the electrode is used to control a motion of an anthropomorphic robotic hand.

In some embodiments, the electrode can quantify muscular strength for neurological assessments.

The polymer composite can be used as a dry contact electrode for detecting electrical signals of the brain. In this application, an electroencephalogram (EEG) signal can be generated. In some embodiments, a perturbed EEG signal is generated through the generation of biopotential of an optic nerve, by opening and closing eyes. In other embodiments, a perturbed EEG signal is generated through the generation of an auditory stimuli.

To improve penetration of the electrode to a subject's head for contact with the scalp, a 2D array can be printed on a surface of the electrode. In some embodiments, an array of vertical pillars with a height of about 2 mm and a diameter of about 1 mm was printed. In other embodiments, the array has an inter-pillar spacing of about 5 mm.

Also provided herein is a method of preparing a polymeric composition and/or polymer composite as defined herein, the method comprising a step of mixing an electrically conductive polymer solution with a sugar alcohol.

In some embodiments, the method comprises contacting the electrically conductive polymer and sugar alcohol mixture with an elastomer.

In some embodiments, the method of preparing or fabricating a polymer composite, comprises:
a) mixing PEDOT:PSS with a sugar alcohol to form a first mixture;
b) mixing the first mixture with WPU to form a second mixture; and
c) curing the second mixture in order to form the polymer composite.

In some embodiments, the first mixture is mixed for at least 30 min. In other embodiments, the mixing is for at least 40 min, 50 min, or 60 min. In other embodiments, the mixing is performed at room temperature, or from about 15° C. to about 40° C. The first mixture can be an aqueous mixture formed in an aqueous medium.

The term “aqueous medium” used herein refers to a water based solvent or solvent system, and which comprises of mainly water. Such solvents can be either polar or non-polar, and/or either protic or aprotic. Solvent systems refer to combinations of solvents which resulting in a final single phase. Both ‘solvents’ and ‘solvent systems’ can include, and is not limited to, pentane, cyclopentane, hexane, cyclohexane, benzene, toluene, dioxane, chloroform, diethylether, dichloromethane, tetrahydrofuran, ethyl acetate, acetone, dimethylformamide, acetonitrile, dimethyl sulfoxide, nitromethane, propylene carbonate, formic acid, butanol, isopropanol, propanol, ethanol, methanol, acetic acid, ethylene glycol, diethylene glycol or water. Water based solvent or solvent systems can also include dissolved ions, salts and molecules such as amino acids, proteins, sugars and phospholipids. Such salts may be, but not limited to, sodium chloride, potassium chloride, ammonium acetate, magnesium acetate, magnesium chloride, magnesium sulfate, potassium acetate, potassium chloride, sodium acetate, sodium citrate, zinc chloride, HEPES sodium, calcium chloride, ferric nitrate, sodium bicarbonate, potassium phosphate and sodium phosphate. As such, biological fluids, physiological solutions and culture medium also fall within this definition. In most embodiments, the aqueous solution is water. In some embodiments, the aqueous solution is deionised water. In some embodiments, the aqueous solution is Millipore water.

In some embodiments, the second mixture is mixed for at least 30 min. In other embodiments, the mixing is for at least 40 min, 50 min, or 60 min. In other embodiments, the mixing is performed at room temperature, or from about 15° C. to about 40° C.

In some embodiments, the second mixture is cured by drop casting the second mixture on a surface. In other embodiments, the second mixture is cured by spin coating the second mixture on a surface. In other embodiments, the curing is performed under heating. In other embodiments, the second polymer is heated from about 50° C. to about 100° C., about 50° C. to about 90° C., about 50° C. to about 80° C., or about 50° C. to about 70° C. In other embodiments, the curing is performed for at least 1 h. In other embodiments, the mixing is for at least 1.5 h, 2 h, or 3 h.

Towards this end, the solvent is removed to form the polymer composite. Advantageously, the curing occurs at a relatively low temperature and without curing agents and/or surfactants.

The aqueous medium is removed via evaporation to give a polymer composite as a gel-like matrix containing the disclosed components.

Also provided herein is the use of an electrical device as defined herein for monitoring of a potential on a subject. Provided herein is the use of the electrical device for measuring an ECG, EMG or EEG signal.

In the embodiments that follows, the components of PEDOT:PSS, waterborne polyurethane (WPU) and D-sorbitol are used as an example to showcase the polymer composite of the present invention. The abbreviation PWS is used to represent the blend of PEDOT:PSS, WPU, and D-sorbitol. However, the skilled person should note that the polymer composition and/or composite is not limited to such combination.

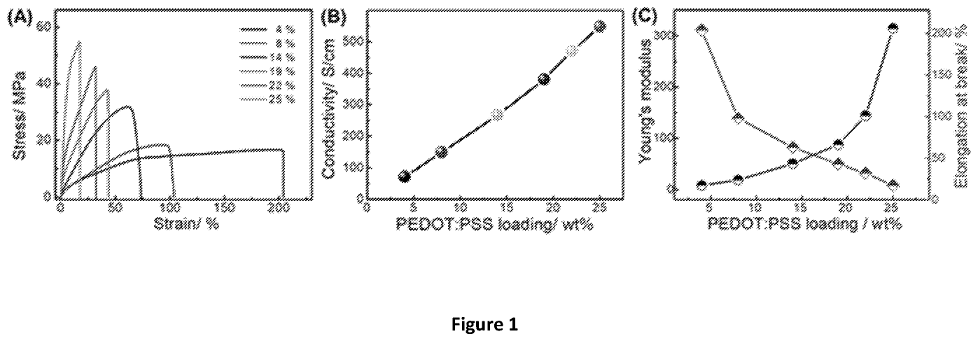

FIG. 1A presents the stress/strain properties of the PEDOT/WPU/S blends with different PEDOT:PSS loadings. The Young's modulus and elongation at break are plotted versus the PEDOT:PSS loading in FIG. 1B. At a low PEDOT:PSS loading of 4 wt %, the blend film has a large elongation at break of 205% and low Young's modulus of 2 MPa. With the increasing of the PEDOT:PSS loading, the elongation at break decreases while the Young's modulus increases. At the PEDOT:PSS loading of 19 wt %, the elongation at break decreases to 43%, while the Young's modulus increases to 85 MPa. Because the skin deformation for human motion in daily life is usually less than 30%, the PEDOT:PSS loading of 19 wt % is mainly employed.

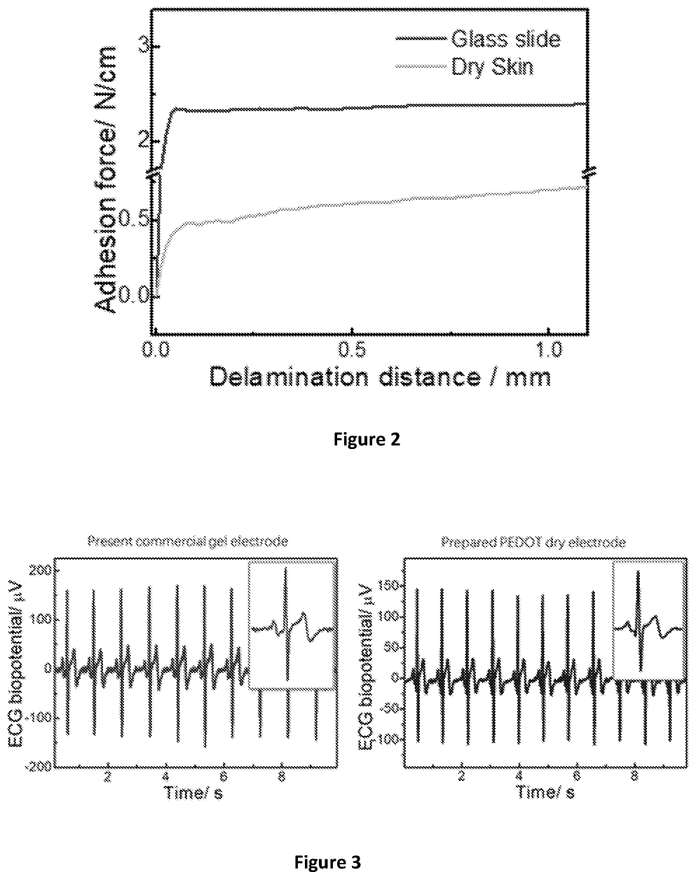

At this optimal composition, the adhesion forces of PEDOT film dry electrode on dry skin and glass are 0.55 and 2.4 N/cm, respectively (FIG. 2). The PEDOT film can attach tightly on skin, even the rough skin of wrist. The good stretchability and adhesion indicate application of PEDOT:PSS/WPU/S as dry electrode for epidermal biopotential measurement. The biosignal such as ECG, EMG and EEG are employed as models to verify the detection performance of prepared PEDOT film dry electrode for epidermal biosignal monitoring.

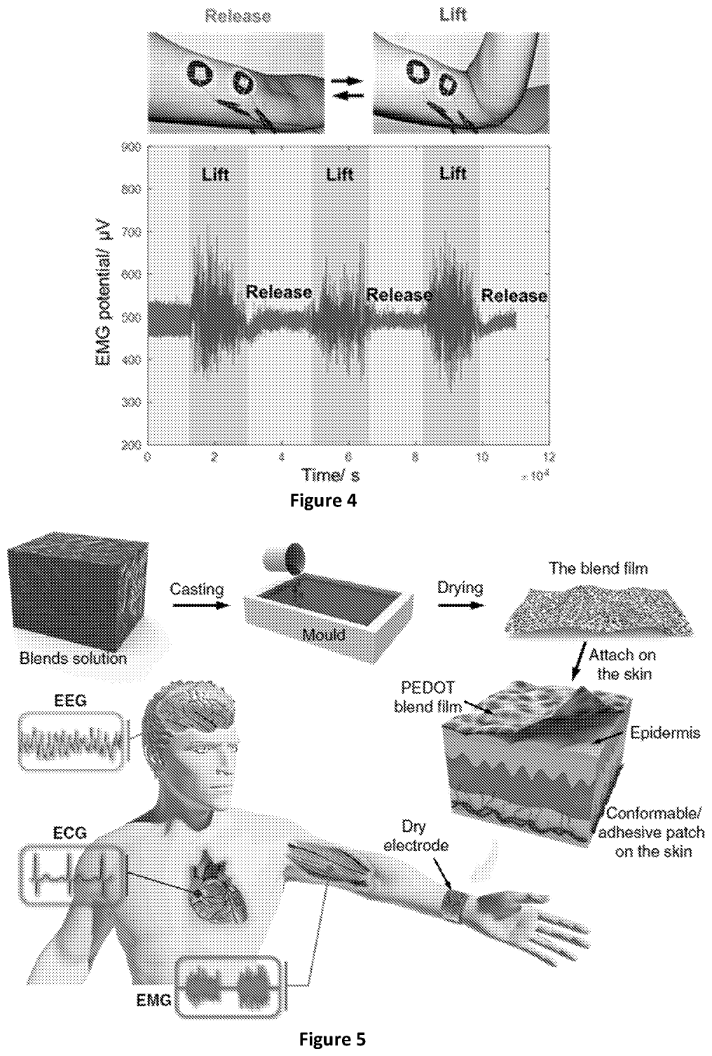

FIG. 3 shows the ECG results using PEDOT film dry electrode and commercial Ag/AgCl gel electrodes. The performance of PEDOT dry electrode is comparable to the standard clinic gel electrode, showing high-quality ECG signal with clear elements of the PQRST waveform. The PEDOT blend films can be also used as a dry electrode of electromyography (EMG) measurement that detects the electric potential generated by muscle. A PEDOT film dry electrodes was placed on the upper arm or forearm of a volunteer to record the EMG signal at different muscle contraction levels (FIG. 4). The EMG signal is record in the range of 1-20 KHz. Potentials were detected when the bicipital muscle was contract or expand, and the potential was almost zero when there was no muscle movement. The signal clearly indicates the muscle activities. The EMG signal using PEDOT dry electrode is almost the same as that using commercial Ag/AgCl gel electrode.

The prepared PEDOT film electrode shows good stretchability, self-adhesiveness and highly conductivity. The blend films can adapt to skin even during body movement and shows low impedance. The PEDOT film electrode are studied as dry electrodes for ECG, EMG and EEG on epidermal skin. They can give rise to high-quality signals and be used for long-term biomedical monitoring. This study indicates that the PEDOT dry electrodes can be used particularly for long-term biopotential monitoring that cannot be achieved by the conventional gel electrodes.

A detailed description of the invention is laid out below.

The PWS electrodes have high conductivity, high mechanical stretchability, excellent adhesiveness to skin and excellent biocompatibility. They are different from other dry electrodes in literature. Nanocomposites with conductive nanofillers in the elastomer matrix can have high stretchability and high conductivity, and they have been studied as dry electrodes for epidermal biopotential measurement. However, the nanocomposite dry electrodes usually give rise to much higher electrode-skin impedance than the PWS electrode because the conductive nanofillers are the minority in the nanocomposites and their effective contact area to skin is thus actually very small. In addition, they are usually not adhesive, and thus high motion artifacts can be observed. Another concern is the possible toxicity of the nanofillers. The PWS blends are also different from the stretchable PEDOT:PSS composites reported in the literature. Stretchable PEDOT:PSS composites were obtained by adding additives. For example, one study found that ionic liquids can significantly increase the stretchability and conductivity of PEDOT:PSS. However, the stretchable PEDOT:PSS composites are not adhesive. They can give rise to high motion artifacts due to the poor skin-electrode contact during body movement. In addition, additives like ionic liquids are toxic, so that PEDOT:PSS added with ionic liquids cannot be used for epidermal biopotential measurement. Although other stretchable PEDOT:PSS composites were used as the dry electrodes, they are not adhesive and thus give rise to high noise during body movement. Some soft adhesive electrodes were reported in literature. For instance, ultrathin electrodes can be adhesive to skin. But they are difficult to handle, and high noise was observed during body movement. Apart from dry electrodes, conductive hydrogels were investigated as adhesive electrodes as well. Because they are wet electrodes, the water vaporization from the hydrogels can induce signal decay and noise. They are not suitable for long term use as well.

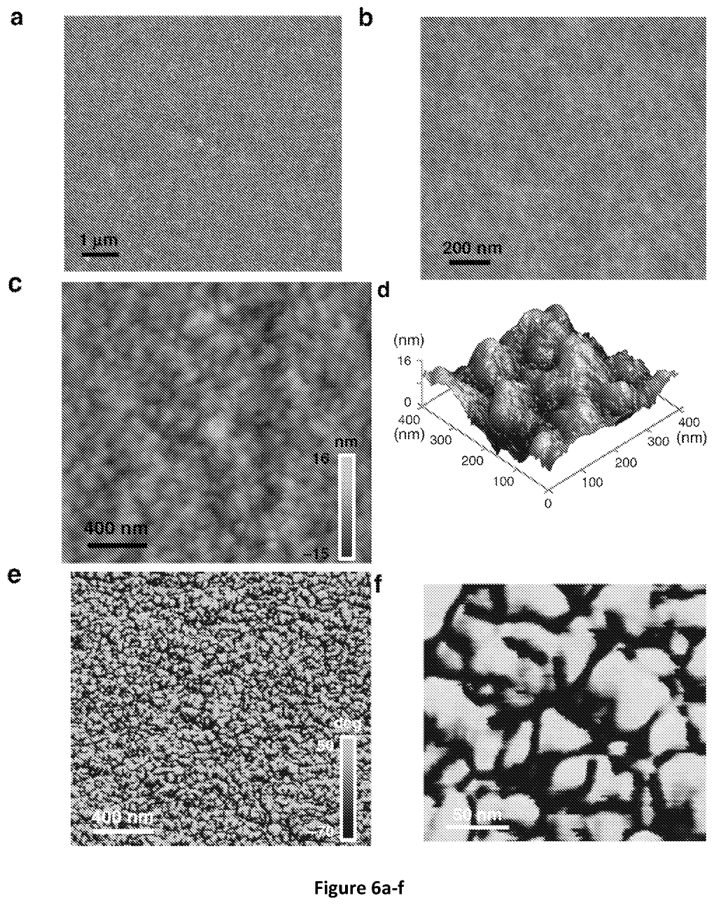

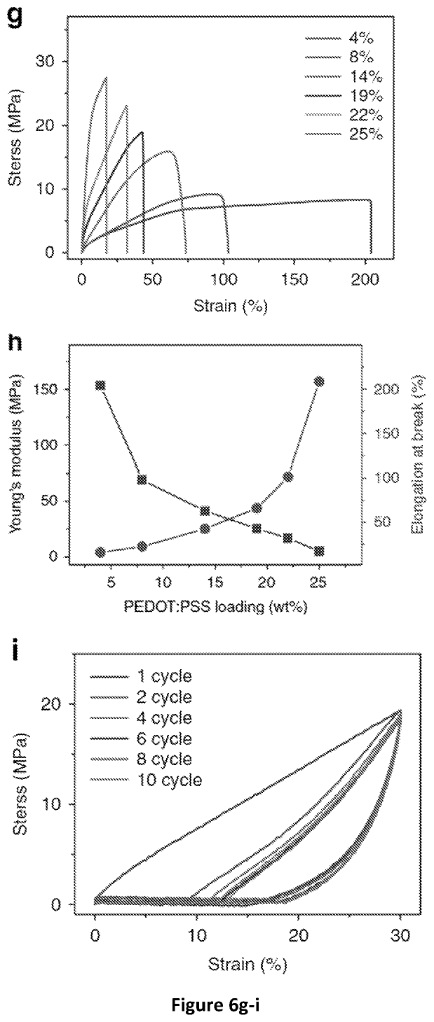

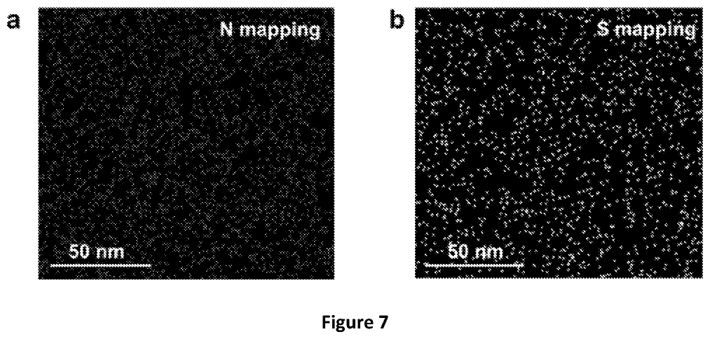

The fabrication process of the self-adhesive dry electrode is illustrated in FIG. 5. Although PEDOT:PSS is intrinsically conductive, it has very limited stretchability and is not adhesive. Nonionic WPU can mix well with PEDOT:PSS solution and improve the stretchability of the PEDOT:PSS film. D-sorbitol is further blended into the mixture to further increase its stretchability. Further advantageously, it was unexpectedly found that D-sorbitol can enhance the adhesiveness of the polymer film on the substrates. Uniform blend films can be prepared by casting aqueous solution consisting of PEDOT:PSS, WPU, and D-sorbitol. The PWS blend films are then investigated as adhesive and stretchable dry electrodes for epidermal biopotentials, including ECG, EMG, and EEG (FIG. 5). The PWS films were characterized by scanning electron microscopy (SEM) and atomic force microscopy (AFM). The SEM image indicates nanoscale grainy morphology (FIG. 6a, b). The grains have a size of ˜100 nm in terms of the topological AFM image (FIG. 6c). The surface roughness is about 16 nm (FIG. 6d). Remarkably, the phase AFM image reveals the presence of two phases in the blend (FIG. 6e, f), because PEDOT:PSS and WPU form a colloidal structure in aqueous solution. This phase structures are supported by the dependence of the phase volume proportion on the loading of PEDOT:PSS in the blends. Higher PEDOT:PSS loading gives rise to a more dark-colored phase. Thus, the dark-colored phase is dominant with PEDOT:PSS, while the light-color phase is mainly due to WPU. The PEDOT chains form conductive networks in the blend film. The presence of the two continuous phases in the blend film is further supported by the element distribution of nitrogen of WPU and sulfur of PEDOT:PSS as revealed by the energy-dispersive X-ray (EDX) results (FIG. 7). Similar microstructure and element distribution were observed by the cross-section SEM images and EDX as well.

Stress-strain curves of polymer composites with different PEDOT:PSS loadings is shown in FIG. 6g. FIG. 6h shows Young's modulus and elongation at break of polymer composites with respect to the PEDOT:PSS loading. FIG. 6i shows tensile stress-strain curves of polymer composite in the first 10 cycles. The tensile speed was 50 mm/min. The PEDOT:PSS loading was 19 wt % for FIG. 6a-f, and i.

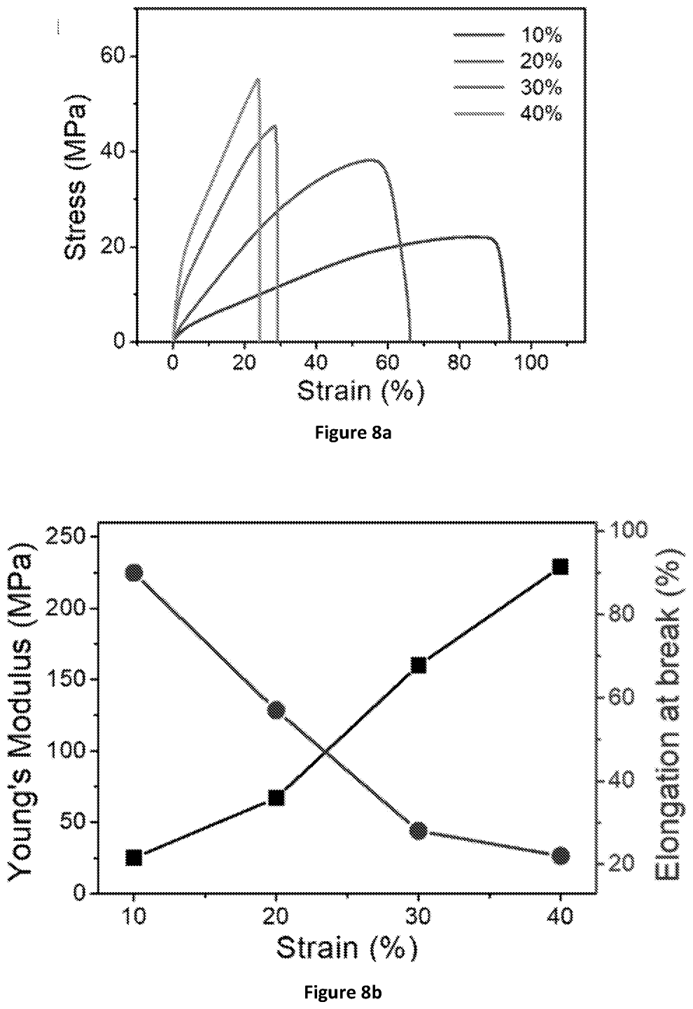

The stress-strain curves of PWS films and PEDOT:PSS/WPU (PW) films without D-sorbitol are shown in FIG. 6g and FIG. 8a respectively. With the increase of the PEDOT:PSS loading, the elongation at break decreases while the Young's modulus increases for both PWS and PW films. The elongation at break is about 28% for the PW films with 30 wt % of PEDOT:PSS (FIG. 8b). The addition of 38 wt % D-sorbitol into this blend can increase the elongation at break to 43% (FIG. 6h). This stretchability is on par with the stretchability of the human skin (˜30%). However, a further increase in D-sorbitol loading can cause remarkable moisture absorption, and make the PWS films volatile and hence susceptible to breaking. Therefore, the optimal loading of D-sorbitol in the blend is found to be about 38 wt %. The PWS films with the optima WPU and D-sorbitol loadings can be stretched repeatedly. As shown in FIG. 6i, although hysteresis can be observed in the first stress-strain cycle, the stretchable behavior becomes stable in the subsequent cycles.

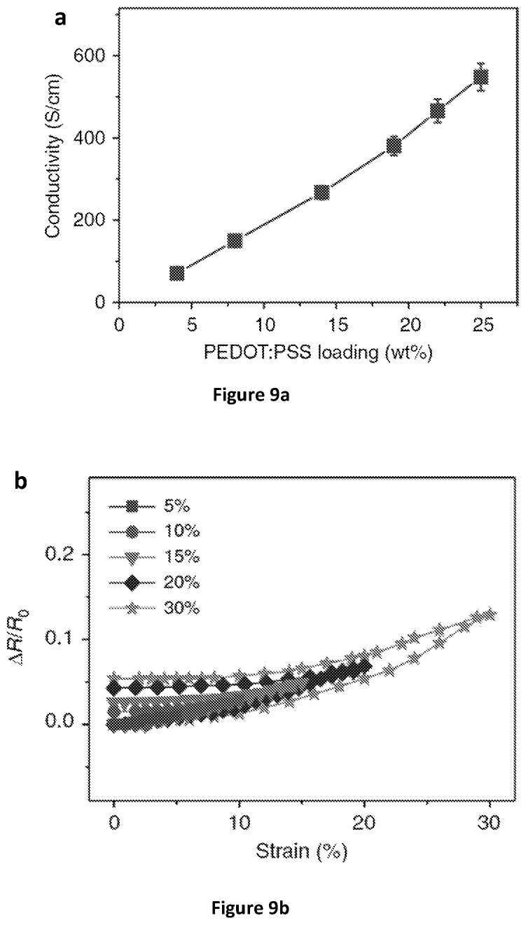

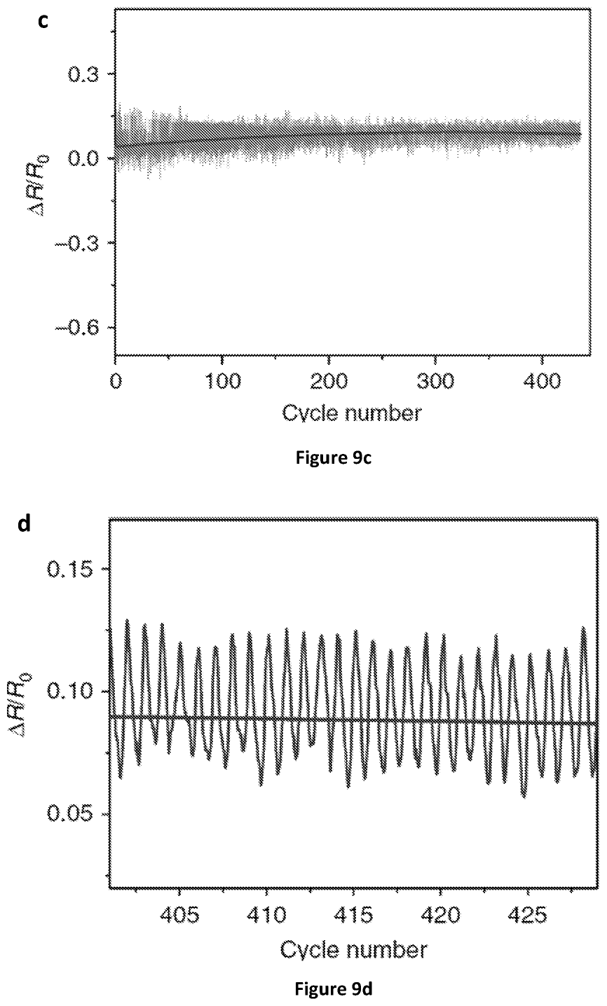

The conductivity of the PWS films depends on the PEDOT:PSS loading as well. The conductivity increases almost linearly from 72 to 545 S/cm when the PEDOT:PSS loading is increased from 4 to 25 wt % (FIG. 9a). This is consistent with the continuous phase structure of PEDOT:PSS in the PWS films. If PEDOT:PSS is instead dispersed as a minority phase in the matrix of WPU, the conductivity of the PWS film should drastically increase until the PEDOT:PSS loading reaches the percolation threshold. Since the skin deformation for human motion in daily life is usually less than 30%, the PWS films with the PEDOT:PSS loading of 19 wt % are investigated for the application as a dry electrode. In the strain range of 30%, the resistance variation is less than 5.5% (FIG. 9b). The resistance in the first three cycles remains almost the same. In the repeated stretching and releasing cycles, the PWS electrodes exhibit stable conductivity. After 440 stretching/releasing cycles, the variation of the conductance is ˜6.5% along the horizontal direction of the PWS film (FIG. 9c, d). The PWS film was stretched to the strain of 30% in each cycle, and the tensile speed was 50 mm/min. The conductance variation along the vertical direction can be even smaller.

The PEDOT networks in the PWS films do not remarkably change in the tensile study. The morphology of a PWS film was studied by SEM and phase AFM before and after stretching to a strain of 30%. No remarkable change can be observed by SEM. The phase AFM images indicate the continuous PEDOT networks in the relaxed or stretched PWS film. This small resistance variation with strain is similar to the conductive PEDOT organogels that have continuous PEDOT networks inside.

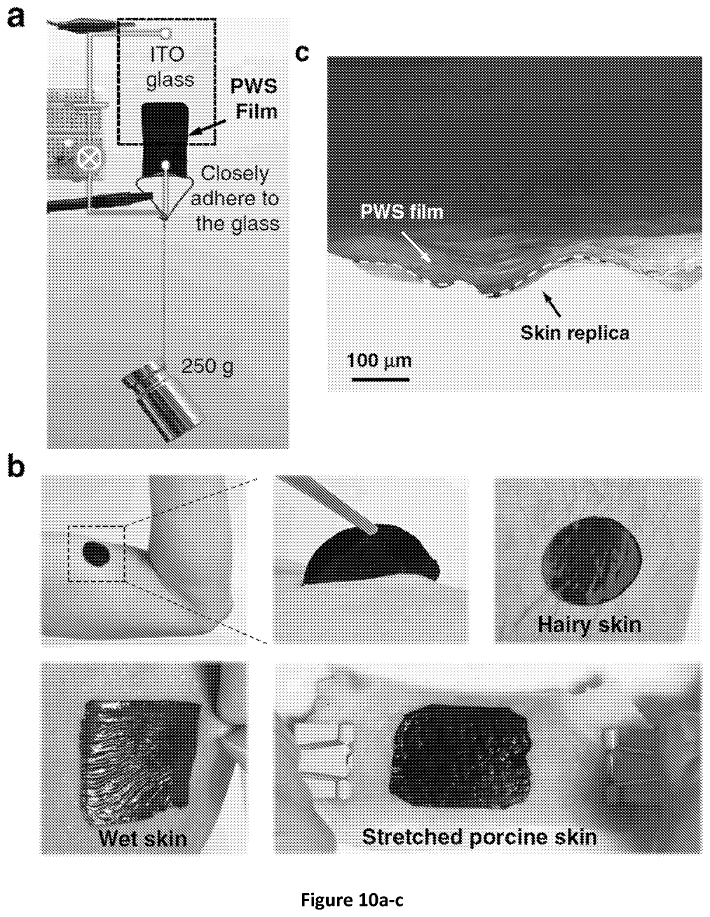

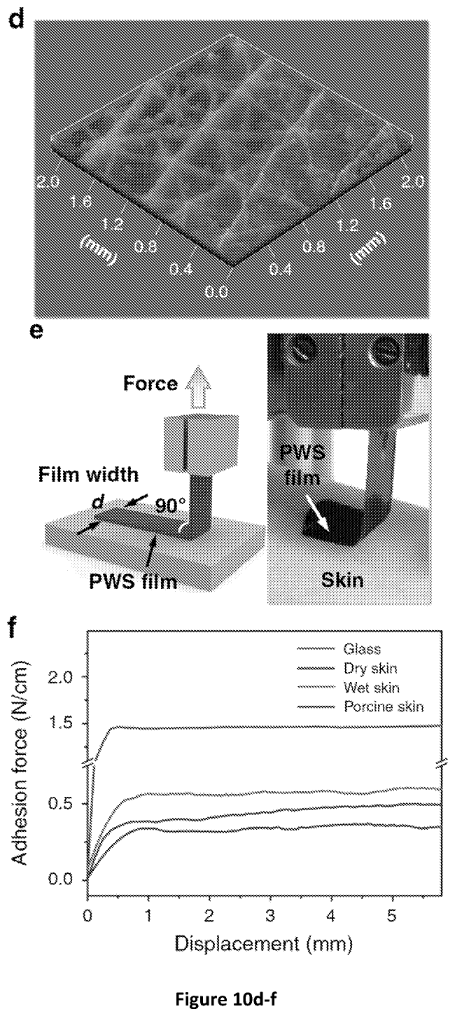

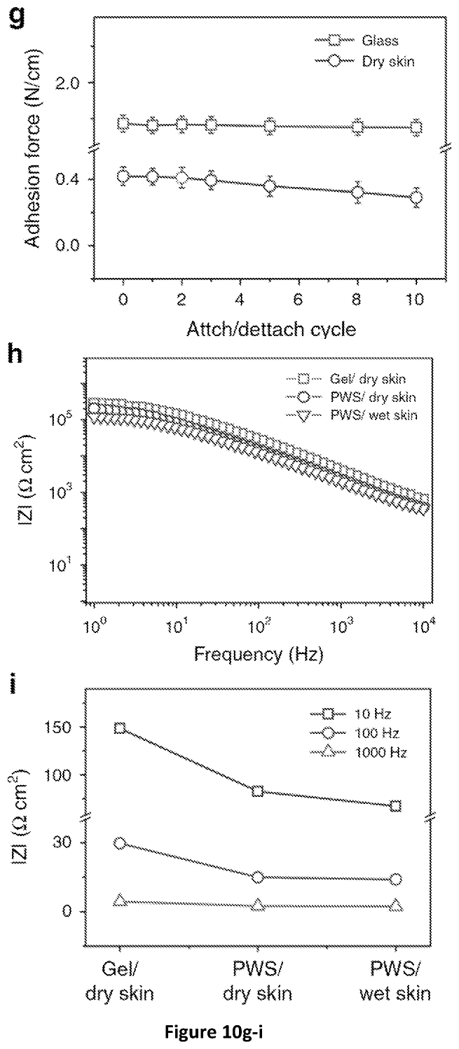

The PWS films exhibit excellent adhesiveness on a glass substrate and skin. A PWS film of 2.5×2.5 cm² and 22±1 μm thick attached on an indium tin oxide (ITO) glass is used in an electrical circuit (FIG. 10a). Even bearing an object of 250 g, the PWS film can attach tightly to the ITO glass and enable the LED in the circuit to work. Moreover, the PWS films can attach tightly on both smooth and hairy skin (FIG. 10b). On the dry and wet skins with substantial wrinkles, the PWS films can adapt to the grooves of the wrinkles and adhere firmly (FIG. 10b). Even on a piece of stretched porcine skin (uniaxial tensile>40%) simulating largely deformed human skin, the PWS film can adapt to the skin deformation and does not delaminate (FIG. 10b). The strain hardly affects the adhesion of the PWS films to the skin. The adhesion force of a pristine PWS film to the skin is 0.43 N/cm. It increases slightly to 0.46 N/cm when the PWS film is stretched to a strain of 30%. The increase in the adhesion force can be attributed to the change in film thickness by strain. The thickness of the pristine PWS film is 20 μm, and it decreases to 15 μm at a strain of 30%. The enhancement in the adhesion force of the stretched PWS film is attributed presumably to the better compliance of thinner film to skin. Even after repeated stretching/releasing cycles, the PWS films still have stable adhesion on glass and skin. They can tightly attach to a wrist that bends and twists vigorously and continuously.

To evaluate the contact of PWS film to skin microscopically, a silicon rubber was used as the skin replica. After placed on the skin replica and pressed for about 3 s, the PWS film could adapt well to the skin replica and exhibited similar skin wrinkle morphology. The cross-section SEM image indicates that the PWS film is conformable to the uneven and curved surface of the skin replica in the sub-millimeter scale (FIG. 10c). The 3D optical image taken with a confocal microscope displays clearly shows the replicated skin wrinkles on the PWS film detached from the skin replica (FIG. 10d). The adhesiveness of the PWS films arises presumably from WPU and D-sorbitol because pristine PEDOT:PSS films are not adhesive. The adhesion mechanism can be attributed to the physical adsorption of PWS to the skin and the mechanical force between them. The surface composition of PWS film is characterized by the IR reflectance spectroscopy. The strong absorption band at around 3300 cm⁻¹ is attributed mainly to the stretching vibration of O—H and N—H group of D-sorbitol and WPU, while the bands at 1725 and 1528 cm⁻¹ can be assigned to the stretching vibration of C═O and the bending vibration of N—H of WPU, respectively. Organic molecules and polymers with O—H, N—H, and C═O groups like cellulose adhesives and polyurethane adhesives can be adhesive due to the strong physical force to substrate. The PWS films with rich O—H, N—H, C═O groups on the surface can have strong physical adsorption to the outermost stratum corneum mostly consisted of keratin and lipids. In addition, the soft PWS films can adapt well with the crevices of the skin, which not only increases the contact area between PWS and skin but also induces adhesive force between them.

The adhesion forces of PWS films to the skin is insensitive to the thickness as the film thickness above 20 μm. The adhesive forces of the PWS films on dry/wet skin and glass are evaluated by the interfacial adhesive force with the standard 90-degree peel testing method (ASTM D2861) (FIG. 10e). The adhesive force (f), f=F(peel force)/d(film width), is plotted against the displacement (L) (FIG. 10f). The PEDOT/WPU (PW) film without D-sorbitol has adhesive forces of 0.12 and 0.18 N/cm on skin and glass, respectively. At the loading of 38 wt % D-sorbitol, the maximum adhesive forces of the PWS films approach 0.41 and 1.44 N/cm on skin and glass, respectively. The further increase of D-sorbitol loading in the PWS film decreases its adhesiveness. The adhesive force is 0.2 N/m on the skin at the D-sorbitol loading of 55 wt %. This force is attributed to the wet and slippery surface of the PWS film caused by the moisture absorption of excess D-sorbitol. The optimal D-sorbitol loading is 38 wt % in terms of the adhesive force. The PWS films are adhesive even to wet skin. A wet skin is obtained by spraying water onto a volunteer's forearm and then removing the large water droplets. The PWS film can have an adhesive force of 0.56 N/cm on this wet skin (FIG. 10f). After ten cycles of attaching/detaching, the adhesion of a PWS film on glass substrate hardly decreases and the adhesion on dry skin only decreases slightly (FIG. 10g). The adhesion reduction on the skin is mainly due to the dirt like sebum. After the contamination is removed by wiping the skin and the PWS electrode with a clinical-grade isopropyl alcohol swab, the adhesion is restored. Hence, the PWS films can be used as a dry electrode repeatedly.

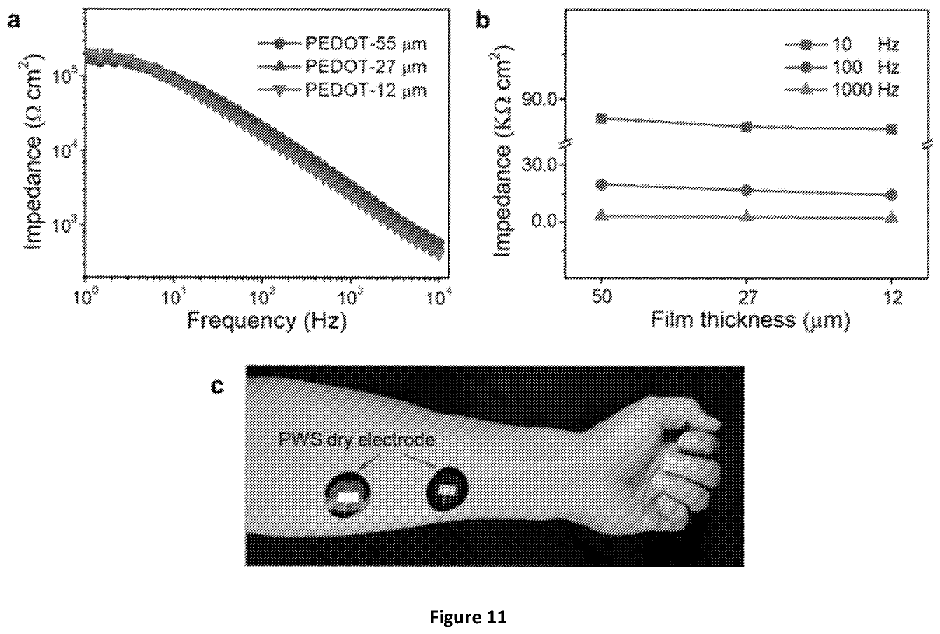

The PWS films have low electrode-skin electrical impedances in the frequency range of 1-104 Hz. Two circular PWS films with a diameter of 3 cm were placed on a volunteer's forearm and their separation was 10 cm. PWS films with the thicknesses of 12, 27, and 55 μm show that the impedances slightly decrease with decreasing film thickness (FIG. 11). This can be attributed to the high conductivity of the PWS films, which is higher than the commercial Ag/AgCl gel electrode by several orders by magnitude. The PWS electrodes exhibits a lower impedance than that of the Ag/AgCl gel electrode (FIG. 10h, i). Their impedances at 10 Hz are 82 KΩ cm² and 148 KΩ cm², respectively. The impedance of the PWS films on skin is much lower than the stretchable dry electrodes in literatures (Table 1).

TABLE 1
Stretchability, impedance, adhesion, and ECG detection performances of various dry electrodes

| | | | | | Applicability | | | |
|---|---|---|---|---|---|---|---|---|
| | | Impedance | ECG | | Static-state | | Dynamic-state | |
| | | at 10 Hz | amplitude | | Noise | | Noise | |
| Electrodes | Stretchability | (κΩ cm²) | (mV) | Adhesion | (mV) | Baseline | (mV) | Baseline |
| PEDOT/Polyimide | Not | ~200 | 0.60 | No | ~0.12 | Normal | Not applicable | |
| | applicable | | | | | | | |
| PEDOT:PSS | Not | 400 | 0.80 | No | ~0.30 | Wander | Not applicable | |
| Coated paper | applicable | | | | | | | |
| PEDOT film/ | 36% | Not | 0.35 | No | ~0.08 | Normal | Not applicable | |
| Ag/AgCl | | applicable | | | | | | |
| PEDOT coated | 10% | 400 | 0.80 | No | ~0.40 | Normal | Not applicable | |
| textile | | | | | | | | |
| PEDOT coated | Low | 300 | 0.55 | No | ~0.05 | Wander | Not applicable | |
| textile | | | | | | | | |
| Ag/PDMS with | Very low | Not | 0.76 | No | ~0.10 | Normal | Not applicable | |
| cilia-patterns | | applicable | | | | | | |
| Ag nanowire/ | 50% | 150 | 0.4 | No | 0.05 | Wander | Not applicable | |
| PDMS/MPU | | | | | | | | |
| blend | | | | | | | | |
| Ag nanowire/ | 400% | 500 | 0.20 | Yes | ~0.05 | Wander | Not applicable | |
| PDMS | | | | | | | | |
| Ag particles/ | Low | 105 | 0.25 | Yes | ~0.04 | Normal | 0.10 | Wander |
| Ecoflex | | | | | | | | |
| Carbon | 100% | Not | 0.50 | Yes | ~0.20 | Wander | 0.10 | Wander |
| nanofillers/ | | applicable | | | | | | |
| (PDMS with | | | | | | | | |
| pillars | | | | | | | | |
| Carbon black/ | 100% | Not | 1.2 | Yes | ~0.20 | Normal | Not applicable | |
| PDMS with | | applicable | | | | | | |
| suckers | | | | | | | | |
| Au thin | Not | 500 | 1.77 | Yes | ~0.16 | Normal | Not applicable | |
| film/polyimide | applicable | | | | | | | |
| Au pattern/ | 26% | 600 | 0.02 | Yes | 0.005 | Normal | Not applicable | |
| Ecoflex/Silicon | | | | | | | | |
| Au film/Parylene/ | 20% | 30 | 1.4 | Yes | 0.052 | Normal | Not applicable | |
| PU fiber | | | | | | | | |
| Au pattern/ | 30% | 250 | 0.3 | No | 0.079 | Normal | Not applicable | |
| Parylene | | | | | | | | |
| AuGa/PDMS | 170% | >200 | — | Yes | — | — | Not applicable | |
| Self-adhesive | 43% | 80 | 1.84 | Yes | <0.025 | Normal | <0.038 | Normal |
| PEDOT film | | | | | | | | |

Compared with highly conductive nanocomposite electrodes with metal nanoparticles or nanowires, the PWS electrodes show significantly lower skin-contact impedance, although the conductivity of the latter can be lower than the former. This is because the impedance is mainly related to the electrode-skin contact instead of the conductivity of the electrode material. The effective contact area between the conductive nanofillers of nanocomposites and skin is very small because the nanofillers are the minority with loading of usually <2 vol %. The loading of the nanofillers cannot be too high, because more nanofillers will lower the stretchability/softness and the adhesiveness of the nanocomposites. Those dry electrodes in literature do not have the other merits of the PWS films, such as the mechanical stretchability and the self-adhesiveness. In addition, the impedance of the PWS films on skin hardly changes over a long period. The impedance slightly decreases in the first 10 min after a PWS film is attached to a skin, which mainly arises from the secretion of sweat on the skin. The impedance is then quite stable over time. Therefore, the PWS films can be used as dry electrodes for long-term healthcare monitoring.

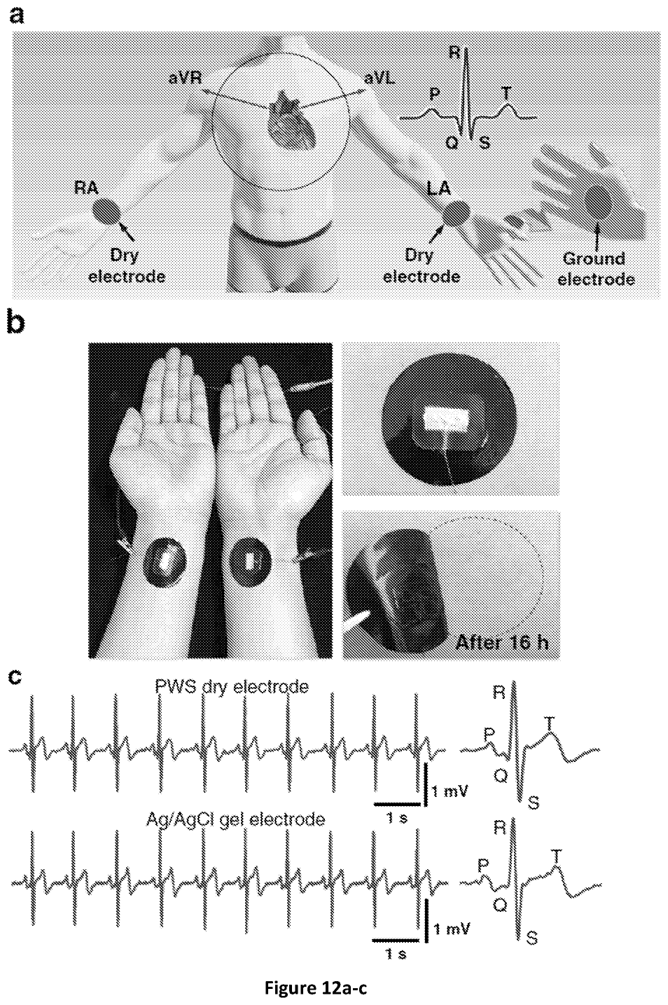

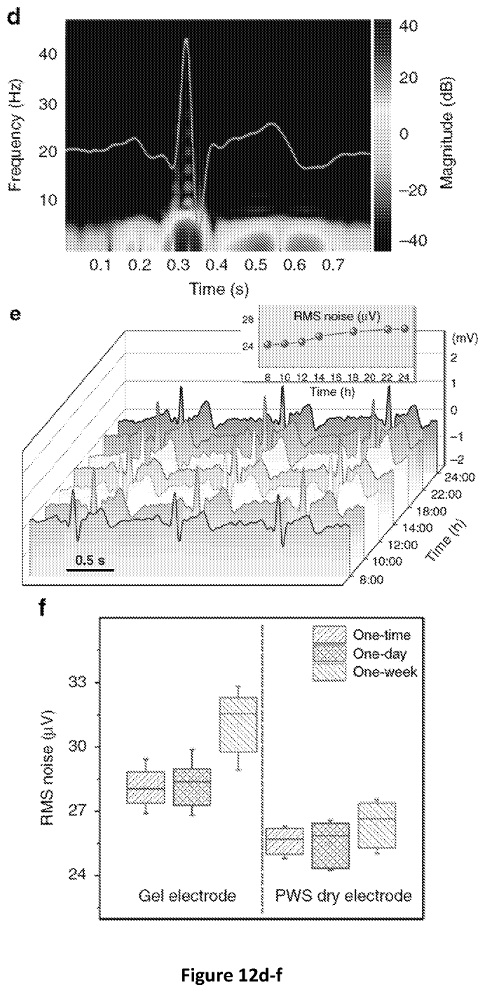

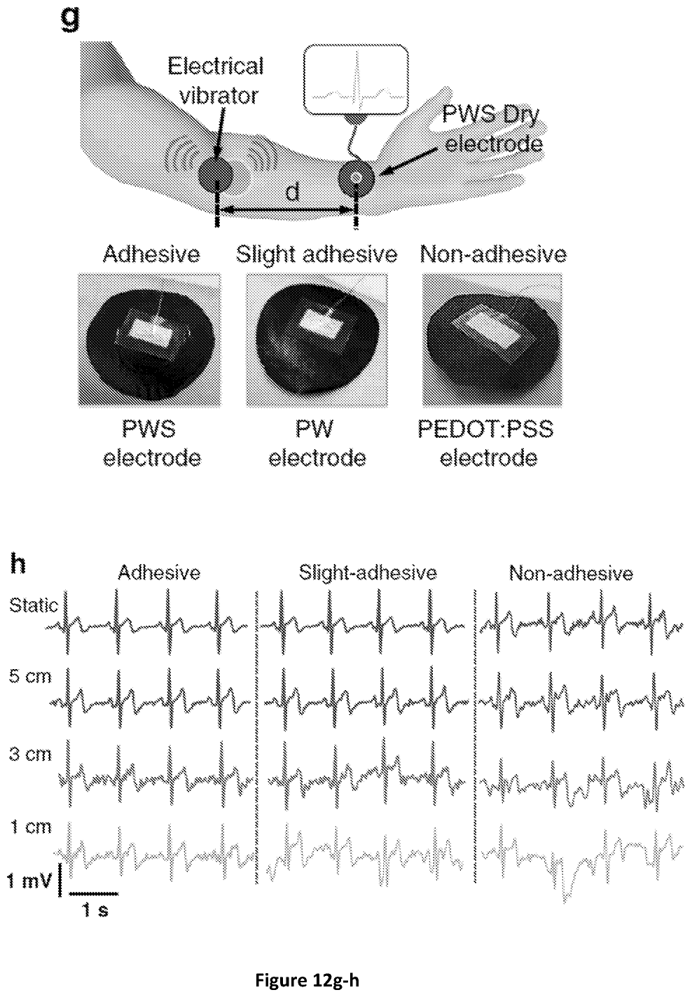

The PWS films can be used as wearable dry electrodes to detect epidermal biopotentials. To record ECG signals, two circular PWS films with a diameter of 3 cm were placed symmetrically on a volunteer's inner wrists of the right and left arms, and another PWS film was attached on the back of the left hand as the ground electrode (FIG. 12a, b). Owing to the biocompatibility and conformability, the PWS electrodes hardly irritate the skin, and no redness is observed even after prolonged use of 16 h (FIG. 12b). The PWS electrodes give rise to high-quality ECG signals with the PQRST waveforms and a peak-to-peak voltage (QRS complex) of 1.84 mV (FIG. 12c). These ECG waveforms are nearly the same as that using the standard Ag/AgCl gel electrodes. In addition, the spectrogram of the ECG pulse in the frequency range of 0-45 Hz is obtained by Fourier transformation (FIG. 12d). The clear frequency identification of PQRST peaks is distinguishable along with the power of the signal in 20-40 dB, and these are critical in clinical settings to diagnose various cardiac signal abnormalities such as congenital heart defects, cardiac arrhythmia, or potential heart failure. The PWS electrodes can be used for long-term healthcare monitoring, as supported by the high-quality ECG signals after continuous use for 16 h (FIG. 12e) and throughout at least 1-month.

The noise of the ECG signal can be evaluated by the root-mean-squared (RMS) analysis, which indicates the fluctuations of the signal over time. The RMS noise picked using the PWS electrodes is about 25 μV, which is even lower than that of Ag/AgCl gel electrodes (28 μV) (FIG. 12f). It is also much lower than that of other dry electrodes in the literature (Table 1). This noise only increases to 27 μV after 1 week (FIG. 12f), while it increases to 32 μV for the Ag/AgCl electrodes. Therefore, the PWS electrodes are much better than the Ag/AgCl electrodes for long term monitoring. The signal quality is also much better than the present dry electrodes using PEDOT or nanocomposites (Table 1).

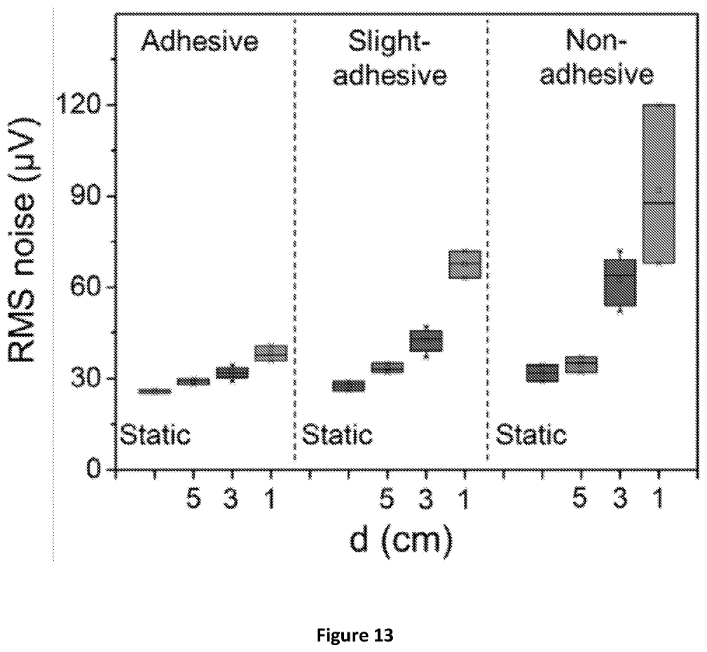

ECG signals were detected during body movement. The body movement was induced by firmly attaching a disc-shaped electromechanical vibrator on the skin (FIG. 12g). The vibrator induced a mean swing amplitude of about 1.5 mm to the skin. The vibration of the skin under the PWS electrode depends on its distance from the vibrator. When the distance (d) is smaller, the skin vibration is more vigorous. ECG signals were recorded at the distance of 5, 3, and 1 cm, respectively (FIG. 12h), and the corresponding noise levels are shown in FIG. 13. The PQRST waveforms are distinguishable without remarkable drift in the baseline, even at the shortest distance of 1 cm. The RMS noise obtained from PWS dry electrodes is below 38 μV, showing high resistance against motion artifacts interferences, which is much better than other dry electrodes (Table 1). The motion artifacts are related to the adhesiveness of the dry electrodes. When slightly adhesive PEDOT:PSS/WPU (PW) films or non-adhesive PEDOT:PSS films are used as the electrodes, significant motion artifacts appear (FIG. 12h). The baseline fluctuations and the noise are even worse when the vibrator is further closer to the electrode. When a PWS film is attached to the skin, it is stretched during the skin movement, such as driven by wrist bending or twisting, which only slightly affects the resistance and adhesion of the PWS electrode. The possible hysteresis in the stress-strain behavior of the PWS film due to the repeated stressing/releasing cycle has little influence on the contact impedance and does not increase the motion artifacts.

The PWS electrodes were further placed on wet skin for ECG testing, as the accurate measurement on the wet and sweaty skin is also a concern for long-term healthcare monitoring. A volunteer's forearm was sprayed with water, and the excess water droplets were removed, leaving a wet skin. The ECG signal on wet skin is almost the same as on dry skin. The ECG signal is not affected when the wrist bends at an angle of 30°, 60°, and 90°. ECG signals can be recorded even when the PWS electrodes attached to the wrist and opisthenar were immersed in water. The PQRST waveforms and stable baseline are observable, with the signal quality saliently higher than that with the commercial Ag/AgCl gel electrodes.

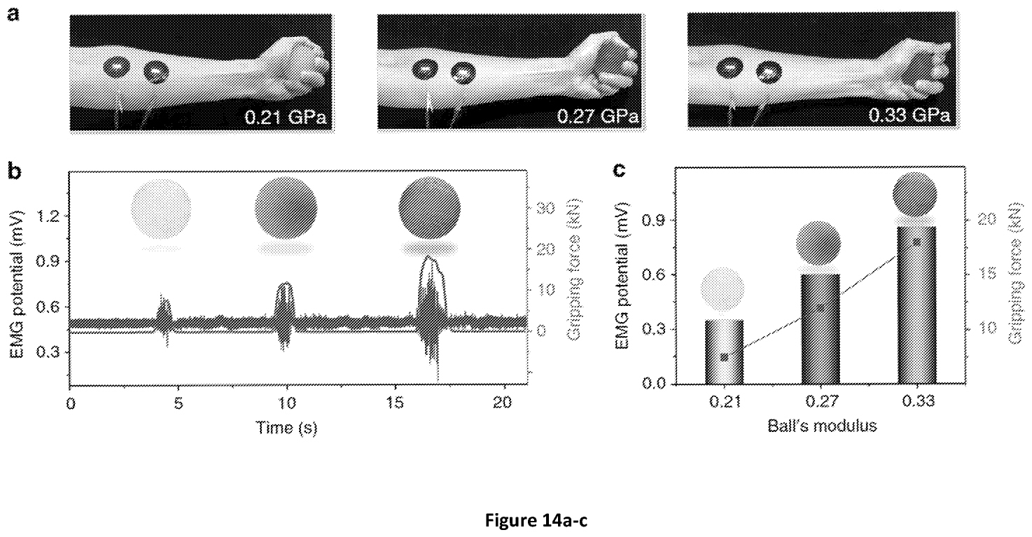

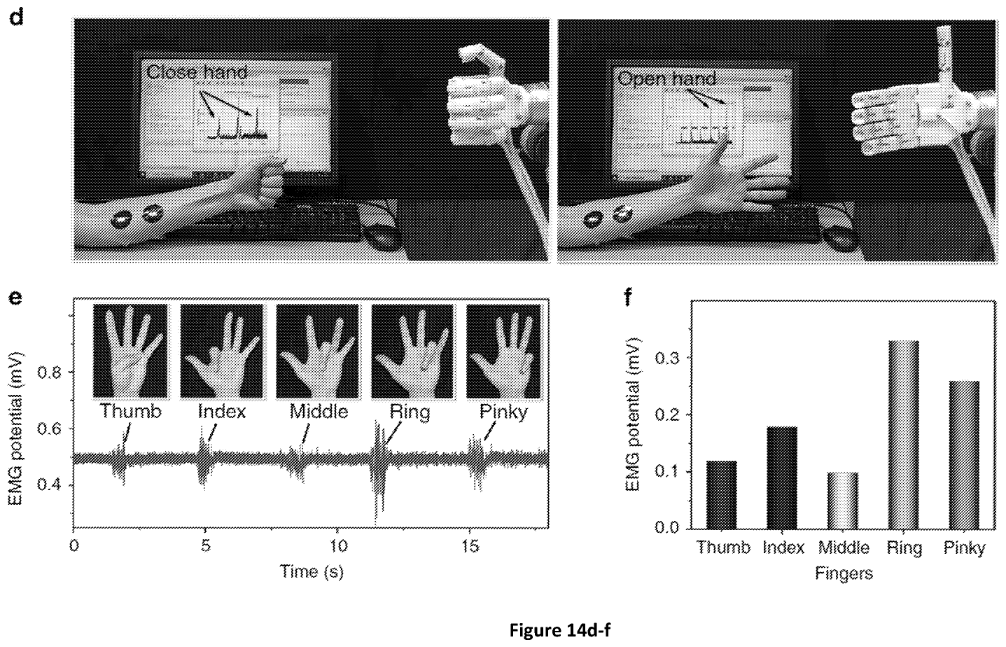

The PWS films can further be used as dry electrodes for EMG that detects the action potential generated by the muscle fibers. As shown in FIG. 14a, two PWS electrodes were placed on the wrist flexors muscles (inner side of the forearm) of a volunteer. When the hand gripes a ball, the wrist flexors contract and generate EMG signals. Different forces are applied to gripe three elastomer balls with the moduli of 0.21, 0.27 and 0.33 GPa, respectively. The corresponding gripping forces imposed on the balls were measured using a commercial optoforce sensor (Optoforce 3-axis force sensor). The peak-to-peak amplitude and the signal intensity are consistent with the gripping force (FIG. 14b, c). The EMG signal using the PWS electrodes is comparable to that with the Ag/AgCl gel electrode. The detection of EMG signals for muscle motion can have essential applications in the human-machine interfaces. For example, the EMG signal of a hand opening/closing from the PWS electrodes can serve as a user interface to control the opening and closing of an anthropomorphic robotic hand in a real-time manner (FIG. 14d). Apart from the significant motion of a bicipital muscle, the PWS electrode can also detect the low amplitude EMG signal produced by a finger performing flexion or extension (FIG. 14e, f).

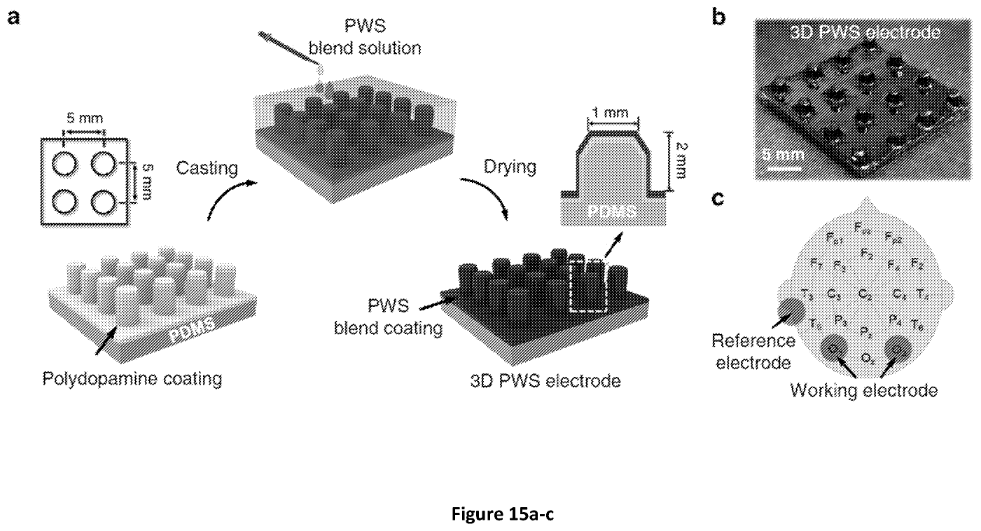

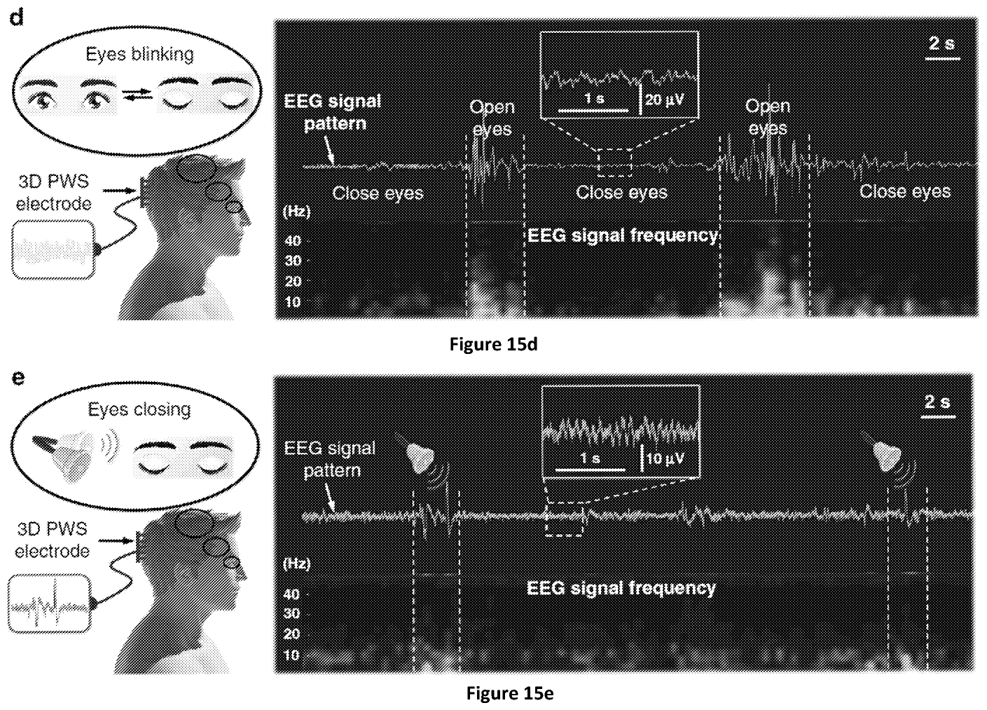

Compared with ECG and EMG, recording high-quality EEG signals is much more challenging due to the weak signal strength in the microvolts range, interference of scalp, and dense hair. In order to achieve decent contact with the hairy scalp, a 3D PWS dry electrode with vertical pillars was fabricated (FIG. 15a). These vertical pillars with a height of 2 mm and a diameter of 1 mm were arranged in a square array with an inter-pillar spacing of 5 mm (FIG. 15b). The pillars do not enhance the adhesiveness but can penetrate through the dense hair to contact the scalp.

To collect the EEG signals at the occipital lobe, two 3D PWS electrodes were mounted at the 01 and 02 sites of the rear head according to the 10-20 system (EEG), and another PWS film electrode was placed behind the ear as a reference electrode (FIG. 15c). To avoid auditory interferences, the volunteer sat in a comfortable position and relaxed by listening to the white noise. The potentials triggered by the optic nerves during the opening and closing of the eyes were detected. The biopotential falls in the frequency ranges of 7-15 Hz during the eye closure, the characteristic alpha waves (FIG. 15d). In contrast, when the eyes are opened, the EEG signals have a broader frequency range. The EEG waves are sensitive to the external sound stimuli. To capture the auditory response, the volunteer sat in a comfortable position and blindfolded to avoid visual interferences. While the eyes were closed, a loud bell was rung at random intervals, and the perturbed EEG signal with different frequency range was captured as responding to the auditory stimuli (FIG. 15e).

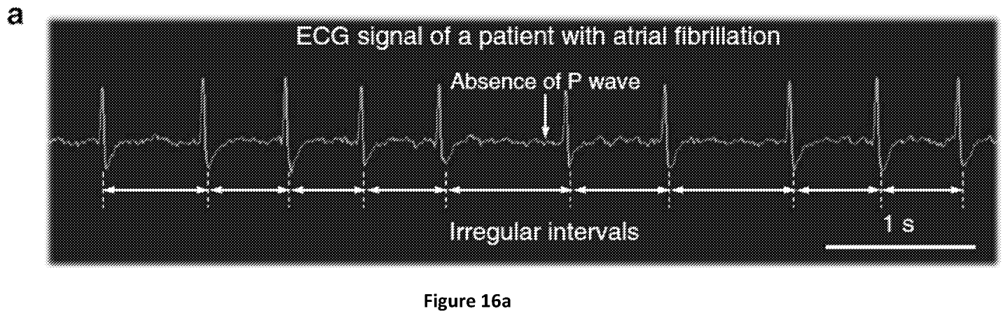

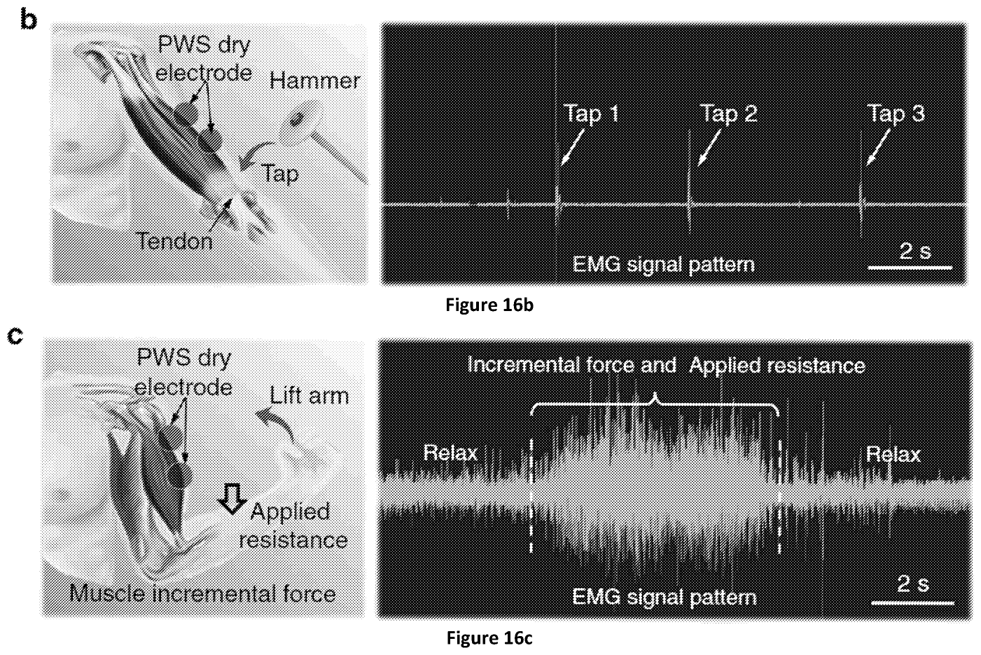

The PWS dry electrodes were further mounted on a patient with atrial fibrillation in a clinic setting to examine the ability of the PWS dry electrodes in identifying electrocardiographic arrhythmia, detecting brief but significant increases in muscle activity during deep tendon reflex testing, and detecting sustained muscle activity during contraction against resistance and during relaxation. The ECG pattern distinctly indicates the absence of typical P peaks and irregular R-R interval (FIG. 16a) consistent with the symptom of atrial fibrillation. In addition, the EMG signals can be used to diagnose the muscle functions of neurological patients. Two PWS dry electrodes were mounted to a patient's upper arm with a separation of 10 cm. When the biceps were triggered by tapping on the biceps tendon, the PWS dry electrodes sensitively recorded an immediate increase in muscle activity induced by contraction (FIG. 16b). In another clinical testing, the patient tried to lift his forearm (under the pulling of biceps contraction) while an incremental external force was applied continuously on his forearm. In this case, an increase in muscle activity persisted during the continuous contraction of the biceps. The PWS dry electrode mounted on the biceps can accurately detect the increment of the EMG signal during continuous contraction and the signal decline in the following relaxation (FIG. 16c). These results suggest the ability of the PWS electrodes to quantify muscular strength for neurological assessments in clinical.

Herein, the blends film of PEDOT:PSS, WPU and D-sorbitol is prepared by solution processing. The resulted PWS films have high conductivity, self-adhesiveness, mechanical flexibility/stretchability and biocompatibility. The PWS film electrodes possess low skin-electrode interfacial impedance and excellent skin-compliance. They can be thus used to acquire high-quality epidermal biopotential signals, including ECG, EMG, and EEG, under various skin conditions. Moreover, the biopotential signals can be immune to motion artifacts. The PWS dry electrodes exhibit remarkably lower skin-electrode impedance and higher signal quality than other dry electrodes in literature. To collect high-quality EEG signals, PWS electrodes with micropillar structures were fabricated to establish secure contact with the scalp through dense hair. The EMG signal using dry electrodes can be used to control the motion of an anthropomorphic robotic hand. To further explore potential applications of these dry electrodes, a clinical study was performed in a patient with atrial fibrillation to identify electrocardiographic arrhythmia, brief but significant increase in muscle contraction during tendon hyper-flexion testing and sustained the increase in muscle contraction against resistance before normalizing during relaxation. The PWS dry electrodes display high adaptability to various conditions and precisely record the epidermal biopotential signals. They have advantages over the commercial Ag/AgCl electrode and other dry electrodes in literature. Therefore, they can be used for long-term healthcare monitoring of patients with regular daily life, rehabilitation, and humanoid robotic instruments.

### Examples

Certain embodiments of the invention will now be described with reference to the following examples which are intended for the purpose of illustration only and are not intended to limit the scope of the generality hereinbefore described.

### Materials

WPU aqueous dispersion (WPU-3-505G) was supplied by Taiwan PU Corporation. The WPU (WPU-3-505G, 39.8 wt %) is a nonionic polyurethane and is used to prepare adhesive blend film with PEDOT and D-sorbitol. PEDOT:PSS aqueous solution (Clevios PH 1000 Lot 2015P0052) was purchased from Heraeus Co. The concentration of PEDOT:PSS was 1.3 wt % in the solution, and the weight ratio of PSS to PEDOT is about 2.5:1. D-sorbitol (97%) and ethylene glycol were obtained from Sigma-Aldrich. Polydimethylsiloxane (PDMS, Sylgard184) and curing agents were obtained from Dow Corning Company. All the chemicals were used as received without further purification.

### Preparation of PWS Films

The PEDOT:PSS solution was mixed with a D-sorbitol aqueous solution and stirred for 30 min at room temperature. Subsequently, ethylene glycol and WPU solution (10 wt %) was added and further stirred for 1 h at room temperature. The PWS films were prepared by drop-casting the above blend solution into a petri dish and dried at 60° C. for at least 2 h. Finally, the resultant PWS films were peeled off after cooling down.

### Preparation of 3D PWS Electrode for EEG Measurement

A flat mold (3 cm×3 cm) with a square array of cylindrical holes (1.5 mm diameter, 2 mm depth) was prepared using polylactic acid by virtue of a 3D printer (LulzBot's TAZ 5 3D printer, Loveland, CO). The PDMS base agent blended uniformly with a curing agent at a weight ratio of 10:1 and cured in an oven at 70° C. for one hour. After demolding, a PDMS substrate with pillar structures was obtained. In order to achieve a wettable surface for the PWS blend solution, a layer of polydopamine was coated on the PDMS substrate by immersing the substrate in the dopamine solution (pH 8.5) for 10 h. The resultant polydopamine-modified PDMS substrate was washed by deionized water and dropped with 4 mL of PWS blend solution consisting of PEDOT:PSS, WPU, and D-sorbitol. After drying at 60° C., a 3D PWS electrode with pillar structures was obtained for the EEG measurement.

### Materials Characterization

The SEM images were collected using a Zeiss Supra 40 field emission scanning electron microscope. The AFM images were obtained using a Veeco NanoScope IV Multi-Mode AFM with the tapping mode. 3D optical microscopic observation was performed on a confocal laser scanning microscope (Carl Zeiss AG, LSM 700, Germany). The thickness of the polymer films was determined with an Alpha 500 step profiler. The impedance spectra were taken with an Autolab impedance analyzer with the dual-electrode method in the ranges of 1-104 Hz. The two electrodes were placed on the forearm with a separation of 10 cm. The conductivities of the polymer films were measured with a four-point probe setup fitted with Keithley 2400 source/meter. In the conductivities shown in figures, the error bars represent the standard error.

### Mechanical Characterization

The tensile measurements were conducted using an Instron Model 5500 Materials Testing System. The load cell is 100 N load cell, and the uniaxial strain was applied at a ramp rate of 1 mm/min. The load cell was calibrated prior to the testing.

### Adhesion Force Characterization

The adhesion force of a PWS film on the substrate was measured through the delamination process using a tensile testing machine (Instron Model 5500 Materials Testing System). A rectangle polymer film of 4×1 cm was laminated on the substrate. The polymer films were then delaminated perpendicularly to the substrate at a rate of 50 mm/min. The adhesion force was calculated in terms of the maximum stable force and the polymer film width. In the plots of the adhesion force, the error bars represent the standard error.

### Biopotential Signal Extraction

The ECG signals were acquired by placing two PWS film electrodes on the inner wrists and a reference electrode on the rear hand. The electrodes were connected to a signal-recording setup processed with a bandpass filter (0.5-150 Hz). The ECG signals were analyzed using the Matlab envelope function. The EMG tests were conducted by mounting two PWS electrodes on the upper arm or forearms and a PWS film as a reference electrode on the rear hand for the signal generated by bicipital or brachioradialis muscle, respectively. For the EMG signals collected for finger flexion and extensions, two PWS electrodes were placed on the forearms. In the EEG measurements, the PWS electrodes with pillars were placed at the 01 and 02 sites according to the 10-20 system of electrode placement on the head. Another PWS film was put on the back of the ear as the reference electrode.

There are two parts to the signal recording setup, including a microcontroller (Arduino UNO microcontroller) and a detector (Muscle SpikerBox Pro). Through potential differences between the working electrodes on the object area and the reference electrodes, the biopotential signals (ECG, EMG, and EEG) are captured by the Spikershield box. The signal processing algorithms are performed on the collected data using Matlab for fundamental signal analysis (Root-Mean-Square/Spectrogram/Fast-Fourier Transform).

Motion artifact measurement of PWS dry electrode during ECG signal recording. A coin button-type cellphone micro vibrator motor with a 1.1 cm2 area is used to generate analogous skin shaking. The vibrator (OEM, JMM181-BY1234BZ3V26L) provided by Yichang Baoyuan Electronics CO. LTD, China) works at a direct voltage of 3 V (/0.1 A), and the rated speed is about 12,000±2500 rpm. The incident skin oscillation amplitude is about 1.5 mm. The vibrator is attached to the inner side of the forearm while the PWS dry electrodes are fixed on the inner wrist. The ECG signal is recorded regularly when the distance between vibrator and PWS electrode is changed to 5, 3, and 1 cm, respectively. The RMS analysis of the ECG signal is performed for evaluating the signal noise and resistance against motion artifact.

As used herein, “and/or” refers to and encompasses any and all possible combinations of one or more of the associated listed items, as well as the lack of combinations when interpreted in the alternative (or).

As used in this application, the singular form “a,” “an,” and “the” include plural references unless the context clearly dictates otherwise. For example, the term “an agent” includes a plurality of agents, including mixtures thereof.

Throughout this specification and the statements which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

Those skilled in the art will appreciate that the invention described herein in susceptible to variations and modifications other than those specifically described. It is to be understood that the invention includes all such variations and modifications which fall within the spirit and scope. The invention also includes all of the steps, features, compositions and compounds referred to or indicated in this specification, individually or collectively, and any and all combinations of any two or more of said steps or features.

1. A polymer composite, comprising:
a) poly(ethylenedioxythiophene):poly(styrenesulfonate) (PEDOT:PSS);
b) waterborne polyurethane (WPU); and
c) a sugar alcohol;
wherein the sugar alcohol is selected from the group consisting of glycerol, erythritol, threitol, arabitol, xylitol, ribitol, mannitol, sorbitol, galactitol, fucitol, iditol, inositol, volemitol, isomalt, maltitol, lactitol, maltotriitol, maltotetraitol, polyglycitol, or a combination thereof; and
wherein the sugar alcohol is about 20 wt % to about 50 wt % of the polymer composite.

2. The polymer composite according to claim 1, wherein a ratio of PEDOT to PSS is about 2.5:1 w/w.

3. The polymer composite according to claim 1, wherein PEDOT:PSS is about 4 wt % to about 25 wt % of the polymer composite.

4. The polymer composite according to claim 1, wherein WPU is about 37 wt % to 58 wt % of the polymer composite.

5. The polymer composite according to claim 1, wherein the sugar alcohol is about 38 wt % of the polymer composite.

6. The polymer composite according to claim 1, the sugar alcohol is D-sorbitol.

7. The polymer composite according to claim 1, the polymer composite further comprises ethylene glycol at about 0.2 wt % to 1.2 wt % of the polymer composite.

8. The polymer composite according to claim 1, the polymer composite comprises a homogenous blend of PEDOT:PSS, WPU and sugar alcohol, wherein PEDOT:PSS and WPU each form separate continuous phases in the polymer composite.

9. The polymer composite according to claim 1, when PEDOT:PSS loading is about 19 wt % of the polymer composite, the polymer composite has an elongation at break is about 35% to about 50%.

10. The polymer composite according to claim 1, the polymer composite having a conductivity of about 60 S/cm to about 600 S/cm.

11. The polymer composite according to claim 1, the polymer composite being repeatedly stretchable for at least 400 cycles.

12. The polymer composite according to claim 1, the polymer composite having a stretchability of more than about 40%.

13. The polymer composite according to claim 1, the polymer composite having an adhesion force to a skin of about 0.35 N/cm to about 0.7 N/cm and/or an adhesion force to a glass surface of about 1 N/cm to about 2 N/cm.

14. (canceled)

15. A polymer composite comprising:
a) poly(ethylenedioxythiophene):poly(styrenesulfonate) (PEDOT:PSS) at a ratio of about 2.5:1 w/w;
b) waterborne polyurethane (WPU); and
c) D-sorbitol;
wherein (PEDOT:PSS) is about 4 wt % to about 25 wt % of polymer composite; and
wherein the sugar alcohol is about 20 wt % to about 50 wt % of the polymer composite.

16. An electrical device comprising a polymer composite according to claim 1.

17. The electrical device according to claim 16, having an electrode-skin electrical impedances at 10 Hz of about 70 KΩ cm² to about 100 KΩ cm².

18. The electrical device according to claim 16 for generating an electrocardiogram (ECG) signal, wherein an ECG peak-to-peak voltage is about 1.6 mV to about 2 mV.

19. The electrical device according to claim 16 for generating an electromyogram (EMG) signal, wherein a peak-to-peak amplitude is linearly correlated to an applied force, and wherein a signal intensity is linearly correlated to the applied force.

20. The electrical device according to claim 16 for generating an electroencephalogram (EEG) signal, wherein the EEG signal perturbable by stimulating an optic nerve of a subject and/or an auditory stimuli.

21. A method of preparing or fabricating a polymer composite, comprises:
a) mixing PEDOT:PSS with a sugar alcohol to form a first mixture;
b) mixing the first mixture with WPU to form a second mixture; and
c) curing the second mixture in order to form the polymer composite;
wherein the sugar alcohol is selected from the group consisting of glycerol, erythritol, threitol, arabitol, xylitol, ribitol, mannitol, sorbitol, galactitol, fucitol, iditol, inositol, volemitol, isomalt, maltitol, lactitol, maltotriitol, maltotetraitol, polyglycitol, or a combination thereof; and
wherein the sugar alcohol is about 20 wt % to about 50 wt % of the polymer composite.
